SEQUENCE LISTING (86 sequences)

SEQ ID NO: 1 - protein, 98 aa, synthetic construct
  REGION 1..98: note = CH1 (CR3)
ASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKV

SEQ ID NO: 2 - protein, 98 aa, synthetic construct
  REGION 1..98: note = CH1 (MUT4)
ASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKKV

SEQ ID NO: 3 - protein, 107 aa, synthetic construct
  REGION 1..107: note = CL (CR3)
RTVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 107 aa, synthetic construct
  REGION 1..107: note = CL (MUT4)
RTVAAPSVFIFPPSDEQLKSGTASVTCLLNNFYPREAKVQWKVDNALQSGNSQESVTEQDSKDSTYSLVSTLTLSKADYEKHKVYACEVTHQGLSSPVTKSFNRGEC

SEQ ID NO: 5 - protein, 117 aa, synthetic construct [sequence omitted]

SEQ ID NO: 6 - protein, 113 aa, synthetic construct [sequence omitted]

SEQ ID NO: 7 - protein, 119 aa, synthetic construct
  REGION 1..119: note = VH cetu
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 8 - protein, 107 aa, synthetic construct
  REGION 1..107: note = VL cetu
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 9 - protein, 120 aa, synthetic construct [sequence omitted]

SEQ ID NO: 10 - protein, 107 aa, synthetic construct [sequence omitted]

SEQ ID NO: 11 - protein, 121 aa, synthetic construct
  REGION 1..121: note = VH matu
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYAGRYFDYWGQGTLVTVSS

SEQ ID NO: 12 - protein, 106 aa, synthetic construct [sequence omitted]

SEQ ID NO: 13 - protein, 215 aa, synthetic construct
  REGION 1..215: note = VH-CH1 patri
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKV

SEQ ID NO: 14 - protein, 220 aa, synthetic construct
  REGION 1..220: note = VL-CL patri
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPAVFIFPPSDEQLKSGTASVVCLLKNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 217 aa, synthetic construct
  REGION 1..217: note = VH-CH1 cetu
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKKV

SEQ ID NO: 16 - protein, 214 aa, synthetic construct
  REGION 1..214: note = VL-CL cetu
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVTCLLNNFYPREAKVQWKVDNALQSGNSQESVTEQDSKDSTYSLVSTLTLSKADYEKHKVYACEVTHQGLSSPVTKSFNRGEC

SEQ ID NO: 17 - protein, 218 aa, synthetic construct
  REGION 1..218: note = VH-CH1 trastu
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKKV

SEQ ID NO: 18 - protein, 214 aa, synthetic construct
  REGION 1..214: note = VL-CL trastu
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVTCLLNNFYPREAKVQWKVDNALQSGNSQESVTEQDSKDSTYSLVSTLTLSKADYEKHKVYACEVTHQGLSSPVTKSFNRGEC

SEQ ID NO: 19 - protein, 219 aa, synthetic construct
  REGION 1..219: note = VH-CH1 matu
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYAGRYFDYWGQGTLVTVSSASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKKV

SEQ ID NO: 20 - protein, 213 aa, synthetic construct
  REGION 1..213: note = VL-CL matu
DIQMTQSPSSLSASVGDRVTITCSASSSVTYMYWYQQKPGKAPKLLIYDTSNLASGVPSRFSGSGSGTDYTFTISSLQPEDIATYYCQQWSSHIFTFGQGTKVEIKRTVAAPSVFIFPPSDEQLKSGTASVTCLLNNFYPREAKVQWKVDNALQSGNSQESVTEQDSKDSTYSLVSTLTLSKADYEKHKVYACEVTHQGLSSPVTKSFNRGEC

SEQ ID NO: 21 - protein, 467 aa, synthetic construct
  REGION 1..467: note = Patri-trastu
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSCDKTHTSPPAPAPELLGGPAAPPAPAPAGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKKV

SEQ ID NO: 22 - protein, 467 aa, synthetic construct
  REGION 1..467: note = Trastu-Patri
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKKVEPKSCDKTHTSPPAPAPELLGGPAAPPAPAPAGGQVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKV

SEQ ID NO: 23 - protein, 466 aa, synthetic construct
  REGION 1..466: note = Patri-Cetu
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSCDKTHTSPPAPAPELLGGPAAPPAPAPAGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKKV

SEQ ID NO: 24 - protein, 466 aa, synthetic construct
  REGION 1..466: note = Cetu-Patri
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKKVEPKSCDKTHTSPPAPAPELLGGPAAPPAPAPAGGQVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKV

SEQ ID NO: 25 - protein, 468 aa, synthetic construct
  REGION 1..468: note = Patri-Matu
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSCDKTHTSPPAPAPELLGGPAAPPAPAPAGGQVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYAGRYFDYWGQGTLVTVSSASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKKV

SEQ ID NO: 26 - protein, 468 aa, synthetic construct
  REGION 1..468: note = Matu-Patri
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYAGRYFDYWGQGTLVTVSSASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKKVEPKSCDKTHTSPPAPAPELLGGPAAPPAPAPAGGQVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKV

SEQ ID NO: 27 - protein, 699 aa, synthetic construct
  REGION 1..699: note = Patri-trastu-Fc
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSCDKTHTSPPAPAPELLGGPAAPPAPAPAGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 699 aa, synthetic construct
  REGION 1..699: note = Trastu-Patri-Fc
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKKVEPKSCDKTHTSPPAPAPELLGGPAAPPAPAPAGGQVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 698 aa, synthetic construct
  REGION 1..698: note = Patri-Cetu-Fc
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSCDKTHTSPPAPAPELLGGPAAPPAPAPAGGQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 698 aa, synthetic construct
  REGION 1..698: note = Cetu-Patri-Fc
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKKVEPKSCDKTHTSPPAPAPELLGGPAAPPAPAPAGGQVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 700 aa, synthetic construct
  REGION 1..700: note = Patri-Matu-Fc
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSCDKTHTSPPAPAPELLGGPAAPPAPAPAGGQVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYAGRYFDYWGQGTLVTVSSASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 700 aa, synthetic construct
  REGION 1..700: note = Matu-Patri-Fc
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYAGRYFDYWGQGTLVTVSSASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKKVEPKSCDKTHTSPPAPAPELLGGPAAPPAPAPAGGQVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 34 aa, synthetic construct
  REGION 1..34: note = Formule G linker
  VARIANT 4: note = Xaa can be any naturally occurring amino acid
  VARIANT 8: note = Xaa can be any naturally occurring amino acid
  VARIANT 10..11: note = Xaa can be any naturally occurring amino acid
  VARIANT 14: note = Xaa can be any naturally occurring amino acid
  VARIANT 24..25: note = Xaa can be any naturally occurring amino acid
  VARIANT 28: note = Xaa can be any naturally occurring amino acid
  VARIANT 30: note = Xaa can be any naturally occurring amino acid
  VARIANT 32: note = Xaa can be any naturally occurring amino acid
EPKXCDKXHXXPPXPAPELLGGPXXPPXPXPXGG

SEQ ID NO: 34 - protein, 34 aa, synthetic construct
  REGION 1..34: note = linker
EPKSCDKTHTSPPAPAPELLGGPAAPPAPAPAGG

SEQ ID NO: 35 - protein, 34 aa, synthetic construct
  REGION 1..34: note = linker
EPKSCDKTHTAPPAPAPELLGGPAAPPAPAPAGG

SEQ ID NO: 36 - protein, 34 aa, synthetic construct
  REGION 1..34: note = linker
EPKSCDKTHTSPPAPAPELLGGPGGPPGPGPGGG

SEQ ID NO: 37 - protein, 34 aa, synthetic construct
  REGION 1..34: note = llinker
EPKSCDKTHTSPPAPAPELLGGPAAPPGPAPGGG

SEQ ID NO: 38 - protein, 34 aa, synthetic construct
  REGION 1..34: note = linker
EPKSCDKTHTCPPCPAPELLGGPSTPPTPSPSGG

SEQ ID NO: 39 - protein, 34 aa, synthetic construct
  REGION 1..34: note = Linker
EPKSCDKTHTSPPSPAPELLGGPSTPPTPSPSGG

SEQ ID NO: 40 - protein, 15 aa, synthetic construct
  REGION 1..15: note = hinge
EPKSCDKTHTCPPCP

SEQ ID NO: 41 - protein, 110 aa, synthetic construct
  REGION 1..110: note = CH2 domain
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 42 - protein, 107 aa, synthetic construct
  REGION 1..107: note = CH3 domain
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 217 aa, synthetic construct
  REGION 1..217: note = CH2-CH3
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 232 aa, synthetic construct
  REGION 1..232: note = Fc domain
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 98 aa, synthetic construct
  REGION 1..98: note = CH1 (CC1)
ASTKGPSVFPLAPSSKSTSGGTAALGCLVKDYFPEPVTVSWNSGALTSGVHTFPAVLQSSGLYSLSSVVDVPSSSLGTQTYICNVNHKPSNTKVDKKV

SEQ ID NO: 46 - protein, 98 aa, synthetic construct
  REGION 1..98: note = CH1 (ML1)
ASTKGPSVFPQAPSSKSTSGGTAALGCLVKDYFPEPVTVSWNSGALTSGVHTFPAVLQSSGLYSLVSVVTVPSSSLGTQTYICNVNHKPSNTKVDKKV

SEQ ID NO: 47 - protein, 98 aa, synthetic construct
  REGION 1..98: note = CH1 (WT)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 48 - protein, 107 aa, synthetic construct [sequence omitted]

SEQ ID NO: 49 - protein, 5 aa, synthetic construct
  REGION 1..5: note = CDR1 VH cetuximab
NYGVH

SEQ ID NO: 50 - protein, 16 aa, synthetic construct
  REGION 1..16: note = CDR2 VH cetuximab
VIWSGGNTDYNTPFTS

SEQ ID NO: 51 - protein, 11 aa, synthetic construct
  REGION 1..11: note = CDR3 VH cetuximab
ALTYYDYEFAY

SEQ ID NO: 52 - protein, 11 aa, synthetic construct
  REGION 1..11: note = CDR1 VL cetuximab
RASQSIGTNIH

SEQ ID NO: 53 - protein, 7 aa, synthetic construct
  REGION 1..7: note = CDR2 VL cetuximab
YASESIS

SEQ ID NO: 54 - protein, 9 aa, synthetic construct
  REGION 1..9: note = CDR3 VL cetuximab
QQNNNWPTT

SEQ ID NO: 55 - protein, 95 aa, Homo sapiens
QVQLQESGPGLVKPSETLSLTCTVSGGSISSYWSWIRQPPGKGLEWIGYYYSGSTNYNPSLKSRVTISVDTSKNQFSLKLSSVTAADTAVYYCAR

SEQ ID NO: 56 - protein, 11 aa, Homo sapiens
WGQGTLVTVSS

SEQ ID NO: 57 - protein, 95 aa, Homo sapiens
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLP

SEQ ID NO: 58 - protein, 10 aa, Homo sapiens
FGQGTKLEIK

SEQ ID NO: 59 - protein, 119 aa, synthetic construct
  REGION 1..119: note = General formula VH humanized cetux
  VARIANT 27: note = Xaa is Gly or Phe
  VARIANT 40: note = Xaa is Pro or Ser
  TURN 70: note = Xaa is Ser or Asn
  VARIANT 71: note = Xaa is Val or Lys
  VARIANT 73: note = Xaa is Thr or Asn
  VARIANT 76: note = Xaa is Asn or Ser
  VARIANT 78: note = Xaa is Phe or Val
  VARIANT 80: note = Xaa is Leu or Phe
  VARIANT 82: note = Xaa is Leu or Met
  VARIANT 88: note = Xaa is Ala or Asn
  VARIANT 6: note = Xaa is Glu or Gln
QVQLQXSGPGLVKPSETLSLTCTVSGXSISNYGVHWIRQXPGKGLEWIGVIWSGGNTDYNTPFTSRVTIXXDXSKXQXSXKXSSVTAXDTAVYYCARALTYYDYEFAYWGQGTLVTVSA

SEQ ID NO: 60 - protein, 107 aa, synthetic construct
  REGION 1..107: note = General formula VL humanized cetux
  VARIANT 11: note = Xaa is Gln or Leu
  VARIANT 21: note = Xaa is Gln or Leu
  VARIANT 45: note = Xaa is Lys or Arg
  VARIANT 83: note = Xaa is Ala or Ile
  VARIANT 85: note = Xaa is Thr or Asp
  VARIANT 100: note = Xaa is Gln or Ala
EIVLTQSPDFXSVTPKEKVTXTCRASQSIGTNIHWYQQKPDQSPXLLIKYASESISGVPSRFSGSGSGTDFTLTINSLEAEDXAXYYCQQNNNWPTTFGXGTKLEIK

SEQ ID NO: 61 - protein, 119 aa, synthetic construct
  REGION 1..119: note = hVH1
QVQLQESGPGLVKPSETLSLTCTVSGGSISNYGVHWIRQPPGKGLEWIGVIWSGGNTDYNTPFTSRVTISVDTSKNQFSLKLSSVTAADTAVYYCARALTYYDYEFAYWGQGTLVTVSA

SEQ ID NO: 62 - protein, 119 aa, synthetic construct
  REGION 1..119: note = hVH2
QVQLQESGPGLVKPSETLSLTCTVSGFSISNYGVHWIRQPPGKGLEWIGVIWSGGNTDYNTPFTSRVTISKDTSKNQVSLKLSSVTAADTAVYYCARALTYYDYEFAYWGQGTLVTVSA

SEQ ID NO: 63 - protein, 119 aa, synthetic construct
  REGION 1..119: note = hVH3
QVQLQESGPGLVKPSETLSLTCTVSGFSISNYGVHWIRQSPGKGLEWIGVIWSGGNTDYNTPFTSRVTISKDNSKNQVSLKLSSVTAADTAVYYCARALTYYDYEFAYWGQGTLVTVSA

SEQ ID NO: 64 - protein, 119 aa, synthetic construct
  REGION 1..119: note = hVH4
QVQLQESGPGLVKPSETLSLTCTVSGGSISNYGVHWIRQPPGKGLEWIGVIWSGGNTDYNTPFTSRVTINKDNSKSQVSFKMSSVTAADTAVYYCARALTYYDYEFAYWGQGTLVTVSA

SEQ ID NO: 65 - protein, 119 aa, synthetic construct
  REGION 1..119: note = hVH5
QVQLQQSGPGLVKPSETLSLTCTVSGFSISNYGVHWIRQSPGKGLEWIGVIWSGGNTDYNTPFTSRVTINKDNSKSQFSLKLSSVTAADTAVYYCARALTYYDYEFAYWGQGTLVTVSA

SEQ ID NO: 66 - protein, 119 aa, synthetic construct
  REGION 1..119: note = hVH6
QVQLQESGPGLVKPSETLSLTCTVSGFSISNYGVHWIRQSPGKGLEWIGVIWSGGNTDYNTPFTSRVTISVDTSKNQFSLKLSSVTAADTAVYYCARALTYYDYEFAYWGQGTLVTVSA

SEQ ID NO: 67 - protein, 119 aa, synthetic construct
  REGION 1..119: note = hVH7
QVQLQQSGPGLVKPSETLSLTCTVSGFSISNYGVHWIRQPPGKGLEWIGVIWSGGNTDYNTPFTSRVTINKDNSKSQVSFKMSSVTAADTAVYYCARALTYYDYEFAYWGQGTLVTVSA

SEQ ID NO: 68 - protein, 119 aa, synthetic construct
  REGION 1..119: note = hVH8
QVQLQQSGPGLVKPSETLSLTCTVSGFSISNYGVHWIRQSPGKGLEWIGVIWSGGNTDYNTPFTSRVTINKDNSKSQVSFKMSSVTAADTAVYYCARALTYYDYEFAYWGQGTLVTVSA

SEQ ID NO: 69 - protein, 107 aa, synthetic construct
  REGION 1..107: note = hVL1
EIVLTQSPDFQSVTPKEKVTITCRASQSIGTNIHWYQQKPDQSPKLLIKYASESISGVPSRFSGSGSGTDFTLTINSLEAEDAATYYCQQNNNWPTTFGQGTKLEIK

SEQ ID NO: 70 - protein, 107 aa, synthetic construct
  REGION 1..107: note = hVL2
EIVLTQSPDFQSVTPKEKVTITCRASQSIGTNIHWYQQKPDQSPKLLIKYASESISGVPSRFSGSGSGTDFTLTINSLEAEDAADYYCQQNNNWPTTFGQGTKLEIK

SEQ ID NO: 71 - protein, 107 aa, synthetic construct
  REGION 1..107: note = hVL3
EIVLTQSPDFQSVTPKEKVTITCRASQSIGTNIHWYQQKPDQSPKLLIKYASESISGVPSRFSGSGSGTDFTLTINSLEAEDAATYYCQQNNNWPTTFGAGTKLEIK

SEQ ID NO: 72 - protein, 107 aa, synthetic construct
  REGION 1..107: note = hVL4
EIVLTQSPDFQSVTPKEKVTITCRASQSIGTNIHWYQQKPDQSPKLLIKYASESISGVPSRFSGSGSGTDFTLTINSLEAEDAADYYCQQNNNWPTTFGAGTKLEIK

SEQ ID NO: 73 - protein, 107 aa, synthetic construct; identical to SEQ ID NO: 69
  REGION 1..107: note = hVL based on IGKV3-21*01

SEQ ID NO: 74 - protein, 107 aa, synthetic construct
  REGION 1..107: note = hVL based on IGKV3-11*01
EIVLTQSPATLSLSPGERATLSCRASQSIGTNIHWYQQKPGQAPRLLIKYASESISGIPARFSGSGSGTDFTLTISSLEPEDFAVYYCQQNNNWPTTFGQGTKLEIK

SEQ ID NO: 75 - protein, 119 aa, synthetic construct
  REGION 1..119: note = hVH based on IGHV4-59*01
QVQLQESGPGLVKPSETLSLTCTVSGFSLTNYGVHWVRQPPGKGLEWLGVIWSGGNTDYNTPLTSRLTISKDNSKNQVSLKLSSVTAADTAVYYCARALTYYDYEFAYWGQGTLVTVSS

SEQ ID NO: 76 - protein, 119 aa, synthetic construct
  REGION 1..119: note = hVH based on IGHV3-33*01
QVQLVESGGGVVQPGRSLRLSCAVSGFSLTNYGVHWVRQAPGKGLEWLGVIWSGGNTDYNTPVTSRFTISKDNSKNTVYLQMNSLRAEDTAVYYCARALTYYDYEFAYWGQGTLVTVSS

SEQ ID NO: 77 - protein, 119 aa, synthetic construct
  REGION 1..119: note = VH cetux N>D
QVQLKQSGPGLVQPSQSLSITCTVSGFSLTNYGVHWVRQSPGKGLEWLGVIWSGGNTDYNTPFTSRLSINKDNSKSQVFFKMNSLQSDDTAIYYCARALTYYDYEFAYWGQGTLVTVSA

SEQ ID NO: 78 - protein, 119 aa, synthetic construct
  REGION 1..119: note = VH cetux N>E
QVQLKQSGPGLVQPSQSLSITCTVSGFSLTNYGVHWVRQSPGKGLEWLGVIWSGGNTDYNTPFTSRLSINKDNSKSQVFFKMNSLQSEDTAIYYCARALTYYDYEFAYWGQGTLVTVSA

SEQ ID NO: 79 - protein, 19 aa, synthetic construct
  REGION 1..19: note = signal peptide heavy chain
MELGLSWIFLLAILKGVQC

SEQ ID NO: 80 - protein, 22 aa, synthetic construct
  REGION 1..22: note = signal peptide light chain
MDMRVPAQLLGLLLLWLRGARC

SEQ ID NO: 81 - protein, 119 aa, synthetic construct
  REGION 1..119: note = VH humanized Cetuximab
QVQLQESGPGLVKPSETLSLTCTVSGFSISNYGVHWIRQSPGKGLEWIGVIWSGGNTDYNTPFTSRVTISKDNSKNQVSLKLSSVTAADTAVYYCARALTYYDYEFAYWGQGTLVTVSS

SEQ ID NO: 82 - protein, 232 aa, synthetic construct
  REGION 1..232: note = Fc domain (S239D and I332E)
EPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 217 aa, synthetic construct
  REGION 1..217: note = VH-CH1 humanized Cetuximab Fab
QVQLQESGPGLVKPSETLSLTCTVSGFSISNYGVHWIRQSPGKGLEWIGVIWSGGNTDYNTPFTSRVTISKDNSKNQVSLKLSSVTAADTAVYYCARALTYYDYEFAYWGQGTLVTVSSASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKRV

SEQ ID NO: 84 - protein, 214 aa, synthetic construct
  REGION 1..214: note = VL-CL humanized Cetuximab Fab
EIVLTQSPDFQSVTPKEKVTITCRASQSIGTNIHWYQQKPDQSPKLLIKYASESISGVPSRFSGSGSGTDFTLTINSLEAEDAATYYCQQNNNWPTTFGAGTKLEIKRTVAAPSVFIFPPSDEQLKSGTASVTCLLNNFYPREAKVQWKVDNALQSGNSQESVTEQDSKDSTYSLVSTLTLSKADYEKHKVYACEVTHQGLSSPVTKSFNRGEC

SEQ ID NO: 85 - protein, 698 aa, synthetic construct
  REGION 1..698: note = Heavy chain BMX003-010
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKRVEPKSCDKTHTSPPAPAPELLGGPAAPPAPAPAGGQVQLQESGPGLVKPSETLSLTCTVSGFSISNYGVHWIRQSPGKGLEWIGVIWSGGNTDYNTPFTSRVTISKDNSKNQVSLKLSSVTAADTAVYYCARALTYYDYEFAYWGQGTLVTVSSASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 698 aa, synthetic construct
  REGION 1..698: note = Heavy chain BMX003-011
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKRVEPKSCDKTHTSPPAPAPELLGGPAAPPAPAPAGGQVQLQESGPGLVKPSETLSLTCTVSGFSISNYGVHWIRQSPGKGLEWIGVIWSGGNTDYNTPFTSRVTISKDNSKNQVSLKLSSVTAADTAVYYCARALTYYDYEFAYWGQGTLVTVSSASTKGPSVFPLAPSSKSTSGGTAALGCQVKDYFPEPVTVSWNSGALTSGVHTFPAVLQSSGLYSLVSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0282892 A1
(43) Pub. Date: Sep. 11, 2025

(54) BISPECIFIC ANTIBODIES BINDING TO HER-3 AND TO EITHER HER-2 OR EGFR

(71) Applicants: BIOMUNEX PHARMACEUTICALS, PARIS (FR); INSTITUT NATIONAL DE LA SANTE ET DE LA RECHERCHE MEDICALE, PARIS (FR); UNIVERSITÉ DE MONTPELLIER, MONTPELLIER (FR); INSTITUT RÉGIONAL DU CANCER DE MONTPELLIER, MONTPELLIER (FR)

(72) Inventors: EUGENE ZHUKOVSKY, BETHEL, CT (US); PIERRE-EMMANUEL GERARD, PARIS (FR); CHRISTEL LARBOURET, VALFLAUNES (FR); EMILIA RABIA, BEZONS (FR); THIERRY CHARDÈS, ASSAS (FR); ANDRÉ PELEGRIN, MONTPELLIER (FR)

(21) Appl. No.: 18/842,834

(22) PCT Filed: Mar. 1, 2023
(86) PCT No.: PCT/EP2023/055243

§ 371 Date: Aug. 30, 2024

(30) Foreign Application Priority Data
Mar. 2, 2022 (EP) 22305242.4

(51) Int. Cl. C07K 16/32; A61K 39/00; A61P 35/00; C07K 16/28

(52) U.S. Cl. CPC C07K 16/32; A61P 35/00; C07K 16/2863; A61K 2039/505; C07K 2317/24; C07K 2317/31; C07K 2317/51; C07K 2317/55

(57) ABSTRACT

The present invention relates to bispecific binding molecules, especially antibodies, targeting HER3 and another antigen selected from HER-2 and EGFR antigens, methods for the production of these molecules, compositions, and uses thereof.

### CROSS-REFERENCE TO RELATED APPLICATION

This application is the U.S. national stage application of International Patent Application No. PCT/EP2023/055243, filed Mar. 1, 2023.

### REFERENCE TO SEQUENCE LISTING

The Sequence Listing for this application is labeled “Seq-List-replace.xml” which was created on Mar. 12, 2025 and is 131,180 bytes. The entire content of the sequence listing is incorporated herein by reference in its entirety.

The invention relates to bispecific antigen-binding molecules, especially antibodies, targeting HER-3 and another antigen selected from HER-2 and EGFR, methods for the production of these molecules, compositions, and uses thereof.

### BACKGROUND OF THE INVENTION

The HER family, which includes 4 tyrosine kinase receptors (EGFR/HER1, HER2, HER3 and HER4), activates multiple, partially redundant, interconnected downstream signaling cascades, e.g. MAPK and PI3K/AKT pathways, which are involved in cell proliferation. The shared general structure of HER receptors consists of an extracellular domain, a single span transmembrane domain and an intracellular domain containing the conserved catalytic kinase domain and carboxy terminal tail. A fundamental aspect of signaling in this family is the dimerization of two receptors. It is the homodimerization and heterodimerization of two HER family members and transphosphorylation of their intracellular regions that generates the initial signal leading to activation of numerous downstream signaling pathways. The four members of the HER family are capable of forming 28 homo and heterodimers.

The deregulation of HER signaling through gene amplification or mutation is seen in many human tumors and an abundance of experimental evidence supports the etiologic role of these events in cancer pathogenesis. HER abnormal signaling has been observed in a large number of solid tumors (lung, colorectal, pancreas, etc.). For example, there is evidence that the HER family is deregulated in pancreatic cancers. EGFR is expressed in 45-95% of pancreatic cancer, and expression generally correlates with worse outcome in resected pancreatic cancers. Overexpression of HER2 has also been described in 7-58% of pancreatic cancer and HER2-amplified pancreatic cancers show an atypical metastatic pattern, suggesting that HER2 is likely to be also an important driver of tumorigenesis in pancreatic cancer. In addition, HER3 expression correlates with tumor progression and reduced survival in patients with pancreatic cancer.

Pancreatic cancer is the fourth most common cause of cancer death in Europe with an increasing number of cases every year (+2% in men, +10% in women). It has a very poor prognosis, even when diagnosed early. It is one of the only cancers for which the survival rate has almost not been improved over the past 40 years: survival is inferior to 20% and 5% after 1 and 5 years respectively. Pancreatic cancer remains a rare disease, with as many deaths recorded in the world in 2020 (466,003) as newly diagnosed patients (495,773), due to lack of effective treatments. At present, pancreatic adenocarcinoma (90% of pancreatic cancers) is treated either surgically, by chemotherapy, or a combination of radiation and chemotherapy with limited results. The launch of gemcitabine in 1996 as first line treatment improved the survival without relapse by 1.3 month in median and the overall survival (OS) at one year from 2% to 18%. Since 2005, only two drugs, Tarceva® (erlotinib) and Abraxane® (nab-paclitaxel), have been authorized for pancreatic cancer, despite various clinical trials, involving mainly combinations but few innovations. Erlotinib, that was the first targeted therapy, improved the survival without relapse by only 1 month in median in association with gemcitabine. With regard to HER3 inhibitors, various molecules are being studied and evaluated in clinical trials (phase I/II).

Treatment by means of therapeutic agents targeting HER receptors directly or downstream kinases often faces acquired resistance or is limited by the intrinsic robustness of the signal transduction network.

In such cases, combined therapies have emerged as natural countermeasures although their optimal design is not straightforward and can depend on the tumor or its subtypes, thus requiring prior patient stratification (Fitzgerald J B, Schoeberl B, Nielsen U B, Sorger P K. Systems biology and combination therapy in the quest for clinical efficacy. Nat Chem Biol. 2006; 2:458-66). The current arsenal available to inhibit HER signaling is comprised of small molecule tyrosine kinase inhibitors (TKIs), e.g. lapatinib or erlotinib, and therapeutic antibodies, e.g. cetuximab or trastuzumab. Indeed, antibodies represent a powerful approach that induces immunological effects on top of signaling reduction to help clearing the tumors as opposed to TKIs that are limited to signaling modulation.

Combined antibody-based therapies have been proposed by a number of authors. Targeting HER dimers, in particular EGFR/HER2 heterodimers, by mAb combinations was demonstrated to be advantageous for inhibition of pancreatic tumor growth.

Bispecific antibodies (BsAb) have further been designed, which combine the targets of two mAbs. However, they suffer from complicated designs that usually result in inferior manufacturability and stability, and efficacy of these antibodies could still be improved, since in in vivo pancreatic cancer model tumors continued to grow even while under treatment with bispecific antibodies.

For instance, Liu and colleagues (Liu et al. A Novel Antibody Engineering Strategy for Making Monovalent Bispecific Heterodimeric IgG Antibodies by Electrostatic Steering Mechanism. J Biol. Chem. 2015; 290 (12): 7535-62) have described construction and characterization of a bispecific anti-EGFR and anti-HER2 antibody, in which panitumumab and trastuzumab sequences, respectively, were utilized. The antibody demonstrated improved activity against EGFR+/HER2+ cell lines in vitro and in vivo, however the activity of this antibody still needs to be improved.

MM-111, an anti-HER2/anti-HER3 bispecific antibody developed by Merrimack Pharma (McDonagh et al., 2012; Spiess et al., 2015) has been studied in patients with HER2-positive carcinomas of the distal esophagus, gastroesophageal (GE) junction and stomach. The Phase II clinical trial was stopped due to non-significant results.

Duligotuzumab, which is an anti-EGFR/anti-HER3 bispecific antibody (Schaefer et al., 2011b; Spiess et al., 2015) was compared to cetuximab in combination with FOLFIRI as second-line therapy in patients with metastatic colorectal cancer. In this trial, duligotuzumab showed no benefit over cetuximab in combination with FOLFIRI and was abandoned.

In view of the above, there is still a need for improved bispecific antibody targeting HER receptors, for treating tumors.

### SUMMARY OF THE INVENTION

The inventors have now designed novel bispecific antibodies targeting HER3, and another antigen selected from either EGFR or HER2, useful in the treatment of a variety of cancers.

The invention more particularly provides a bispecific antigen-binding fragment which is capable of simultaneous binding to HER-3 and to another antigen selected from either HER-2 or EGFR antigens, which comprises:
(i) a Fab fragment comprising a VH-CH1 heavy chain associated with a VL-CL light chain of antibody 1 (Ab1), and
(ii) a Fab fragment comprising a VH-CH1 heavy chain associated with a VL-CL light chain of antibody 2 (Ab2),
   wherein the N-terminal end of the VH domain of the Fab fragment of Ab1 is linked to the C-terminal end of the CH1 domain of the Fab fragment of Ab2 through a polypeptide linker,
   wherein one of Ab1 or Ab2 is patritumab or a functional derivative thereof;
   and the other of Ab1 or Ab2 is selected from the group consisting of trastuzumab or a functional derivative thereof, matuzumab or a functional derivative thereof, and cetuximab or a functional derivative thereof.

In a preferred embodiment, Ab2 is patritumab or a functional derivative thereof and wherein Ab1 is selected from the group consisting of trastuzumab or a functional derivative thereof, matuzumab or a functional derivative thereof, and cetuximab or a functional derivative thereof.

In a particular embodiment, the functional derivative of cetuximab is a humanized form comprising VH and VL chain amino acid sequences at least 80% identical to VH and VL chain amino acid sequences of cetuximab, respectively.

In a particular embodiment, the CH1 and CL domains of Ab1 have a sequence different from the CH1 and CL domains of Ab2.

In a particular embodiment, the Fab CH1 domain of one of Ab1 or Ab2 is a mutated domain that derives from the CH1 domain of an immunoglobulin by substitution of the threonine residue at position 192 of said CH1 domain with a glutamic acid and the cognate CL domain is a mutated domain that derives from the CL domain of an immunoglobulin by substitution of the asparagine residue at position 137 of said CL domain with a lysine residue and substitution of the serine residue at position 114 of said CL domain with an alanine residue, and/or wherein the Fab CH1 domain of one or the other of Ab1 or Ab2 is a mutated domain that derives from the CH1 domain of an immunoglobulin by substitution of the leucine residue at position 143 of said CH1 domain with a glutamine and substitution of the serine residue at position 188 of said CH1 domain with a valine residue, and the cognate CL domain is a mutated domain that derives from the CL domain of an immunoglobulin by substitution of the valine residue at position 133 of said CL domain with a threonine residue and substitution of the serine residue at position 176 of said CL domain with a valine residue.

In a particular embodiment, the polypeptide linker sequence comprises or consists of amino acid sequence: EPKX1CDKX2HX3X4PPX5PAPELLGGPX6X7PPX8PX9PX10GG (SEQ ID NO: 33), wherein X1, X2, X3, X4, X5, X6, X7, X8, X9, X10, identical or different, are any amino acid.

The invention also provides a bispecific molecule that comprises two identical antigen-binding arms, each consisting of an antigen-binding fragment as defined above.

In a preferred embodiment, the bispecific molecule of the invention is a full-length antibody comprising two heavy chains and four light chains,
wherein each heavy chain comprises
   a. a Fc region of an immunoglobulin comprising Hinge-CH2-CH3 domains
   b. which Fc region is linked to the Fab VH-CH1 heavy chain of Ab1 by said hinge domain, wherein the hinge domain links the N-terminal end of said CH2 domain with the C-terminal end of CH1 domain of Ab1;
   c. which in turn is linked to Fab VH-CH1 heavy chain of Ab2, by the polypeptide linker sequence, wherein the polypeptide linker sequence links the N-terminal end of said VH domain of Ab1 with the C-terminal end of said CH1 domain of Ab2,
and wherein the four light chains comprise two Fab VL-CL light chains of Ab1 and two Fab VL-CL light chains of Ab2 associated with their cognate heavy chain domains.

In another embodiment, the bispecific molecule comprises a) two heavy chains, each comprising, preferably consisting of SEQ ID NO: 27 and b) four light chains, two comprising, preferably consisting of SEQ ID NO: 14 and the two others comprising, preferably consisting of SEQ ID NO: 18.

In another embodiment, the bispecific molecule comprises a) two heavy chains, each comprising, preferably consisting of, SEQ ID NO: 29 and b) four light chains, two comprising, preferably consisting of SEQ ID NO: 14 and the two others comprising, preferably consisting of SEQ ID NO: 16.

In another embodiment, the bispecific molecule comprises a) two heavy chains, each comprising, preferably consisting of SEQ ID NO: 31 and b) four light chains, two comprising, preferably consisting of SEQ ID NO: 14 and the two others comprising, preferably consisting of SEQ ID NO: 20.

The invention also relates to a method for producing the bispecific molecule of the invention, said method comprising the following steps:
a. Culturing in suitable medium and culture conditions a host cell expressing an antibody heavy chain as defined above, and an antibody light chain as defined above, and
b. recovering said produced antibodies from the culture medium or from said cultured cells.

The invention also relates to the bispecific antigen-binding fragment or the bispecific molecule of the invention, for use as a medicament.

The invention also relates to the bispecific antigen-binding fragment or the bispecific molecule of the invention, for use in treating a cancer, preferably solid tumors such as pancreatic cancer, head and neck cancer, colorectal cancer, breast cancer, or lung cancer, preferably for use in treating pancreatic cancer.

### LEGENDS TO THE FIGURES

FIG. 1. Schematic drawing of a bispecific antibody of the invention.

FIG. 2. Dual engagement of the 2 antigens simultaneously by ELISA. Binding curve showing simultaneous binding of (A) Patri-Trastu-Fc to immobilized HER3-Fc and HER2-His (left panel), and immobilized HER2-Fc and HER3-His (right panel), (B) Patri-Matu-Fc to immobilized HER3-Fc and EGFR-His (left panel), and immobilized EGFR-Fc and HER3-His (right panel) or (C) Patri-Cetu-Fc to immobilized HER3-Fc and EGFR-His (left panel), and immobilized EGFR-Fc and HER3-His (right panel). Half-maximal efficient concentration (EC50) from ELISA binding curves are indicated.

FIG. 3. Biological properties of the bispecific antibodies to inhibit phosphorylation of AKT (pAKT) and ERK (PERK). Cell lines (A) were pre-stimulated with the bispecific antibodies (B), for 20 min before adding a mix of NRG1/EGF ligands for another 10 min. After cell lysis, the expression levels of pAKT and pERK were quantified by HTRF. The TR-FRET signal (665 nm/620 nm emission ratio) was measured on a Pherastar reader relative to the maximal phosphorylation (100%; medium) obtained in NRG1/EGFR-stimulated cells without bispecific antibody. Phosphorylation levels are presented as percentages, ranging from 0% (white) to 100% (black) corresponding to phosphorylation in the absence of BsAbs “culture medium.”

FIG. 4. In vivo Efficacy of bispecific antibodies in Nude mouse models. Tumor growth (left panel) and survival (middle panel) of bispecific antibody-treated mice xenografted with Sw1990 (A) and PDX P2846 (B) PDAC cells. The % of tumor growth inhibition (TGI) at the end of treatment, and the 50%-survival benefit (days) are indicated in the right panel. The grey area corresponds to the duration of the antibody treatment.

FIG. 5. Intratumoral penetration of bispecific antibodies in Sw-1990 xenografts. Immunohistochemistry analysis of Sw1990 tumor penetration by the bispecific antibodies. Bispecific antibodies are labelled using peroxidase-conjugated anti-human Fc. BsAb IRR is a control bispecific antibody targeting CD19 and CD3. A 9%. NaCl solution was used as negative control. Tumor penetration of Patri-Cetu-Fc was visualized globally on the whole tumor slice (left) and under ×40 microscopy (right).

FIG. 6. Anti-tumoral features of the TME are observed in Sw1990-Xenograft pancreatic mouse model after treatment with the bispecific antibodies of the invention. (A) Immunohistochemistry analysis to monitor CD31+ angiogenesis in resected Sw1990 xenografts from treated mice. Microscopic images of Patri-Cetu-Fc-vs BsAb IRR- and NaCl-treated xenograft sections are shown as examples (left panel). The mean size of CD31+ micro-vessels in tumors were quantified using the Qupath software on the whole tumor section. (B) NK cell immunophenotyping by flow cytometry of resected Sw1990 xenografts from BiXAb-treated mice. Dissociated cells were labeled with human L/D, CD45, CD3, CD19, CD49, NKp46, IFNg− and CD107a-specific antibodies. After negative selection of CD45+CD3+CD19+ cells, CD49+ NKp46+NK cells were positively-gated, before CD107a and intracellular IFNg labelling. (C) Analysis of in vivo ErbB degradation. Immunofluorescence microscopic images of EGFR staining of Patri-Cetu-Fc-vs BsAb IRR- and NaCl-treated Sw1990 xenograft section (top panel). Western blot analysis of ErbB receptor expression for each Sw1990 xenograft extract treated with NaCl solution, Patri-Trastu-Fc, Patri-Matu-Fc and Patri-Cetu-Fc, respectively. Tubulin was used as loading control. Western blots were visualized and quantified using the LI-COR Odyssey imaging system (middle panel). Quantification of ErbB expression, normalized to tubulin, in each group of resected xenografts (bottom panel). Each experiment was performed on three Sw1990-xenografted mice. BsAb IRR is a negative control which binds simultaneously to human CD19 and CD3.

FIG. 7. In vivo efficacy of the bispecific antibody Patri-Cetu-Fc vs monospecific antibodies. Tumor growth (top panel) and survival (bottom panel) of treated mice xenografted with Sw1990 cells. All antibodies in different mouse cohorts contained identical molarity of antibodies based on their Fc-content: Patri-Cetu-Fc was used at 17 mg/kg, the monoclonal controls (Patritumab or Cetuximab) at 10 mg/kg, and the combination (Patritumab+Cetuximab) at 5 mg/kg each. The grey area corresponds to the duration of the antibody treatment.

FIG. 8. Binding profiles of BMX003-001 (Patri-Cetu-Fc), BMX003-010 (humanized Patri-Cetu-Fc), and BMX003-011 (humanized Patri-Cetu-Fc modified) in a HER3 antigen binding ELISA (n=1).

FIG. 9. Binding profiles of BMX003-001 (Patri-Cetu-Fc), BMX003-010 (humanized Patri-Cetu-Fc), and BMX003-011 (humanized Patri-Cetu-Fc modified) in a EGFR antigen binding ELISA (n=3).

FIG. 10. Ability of BMX003-001 (Patri-Cetu-Fc), BMX003-010 (humanized Patri-Cetu-Fc), and BMX003-011 (humanized Patri-Cetu-Fc modified) to bind to the HER3 and EGFR proteins expressed on the cell surface of SW-1990 cells, measured using flow cytometry (n=3).

FIG. 11. Biological properties of BMX003-001 (Patri-Cetu-Fc), BMX003-010 (humanized Patri-Cetu-Fc), and BMX003-011 (humanized Patri-Cetu-Fc modified) to inhibit phosphorylation of AKT (A) and ERK (B), in response to growth factors.

FIG. 12. Capacity of BMX003-001 (Patri-Cetu-Fc), BMX003-010 (humanized Patri-Cetu-Fc), and BMX003-011 (humanized Patri-Cetu-Fc modified) to induce NK cell degranulation (expression of CD107a) in presence of HCT-116 cancer cells (2 independent donors, A and B).

FIG. 13. In vivo efficacy study of BMX003-001 (Patri-Cetu-Fc), BMX003-010 (humanized Patri-Cetu-Fc), and BMX003-011 (humanized Patri-Cetu-Fc modified) with SW1990 pancreatic cancer xenograph model (BMX003-01, BMX003-010 and BMX003-011 used at 8.5 mg/kg and BMX control at 17 mg/kg).

### DETAILED DESCRIPTION

### Definitions

The basic structure of a naturally occurring antibody molecule is a Y-shaped tetrameric quaternary structure consisting of two identical heavy chains and two identical light chains, held together by non-covalent interactions and by inter-chain disulfide bonds.

In mammalian species, there are five types of heavy chains: α, δ, ε, γ, and μ, which determine the class (isotype) of immunoglobulin: IgA, IgD, IgE, IgG, and IgM, respectively. The heavy chain N-terminal variable domain (VH) is followed by a constant region, containing three domains (numbered CH1, CH2, and CH3 from the N-terminus to the C-terminus) in heavy chains γ, α, and δ, while the constant region of heavy chains μ and ε is composed of four domains (numbered CH1, CH2, CH3 and CH4 from the N-terminus to the C-terminus). The CH1 and CH2 domains of IgA, IgG, and IgD are separated by a flexible hinge, which varies in length between the different classes and in the case of IgA and IgG, between the different subtypes: IgG1, IgG2, IgG3, and IgG4 have respectively hinges of 15, 12, 62 (or 77), and 12 amino acids, and IgA1 and IgA2 have respectively hinges of 20 and 7 amino acids.

There are two types of light chains: λ and κ, which can associate with any of the heavy chains isotypes, but are both of the same type in a given antibody molecule. Both light chains appear to be functionally identical. Their N-terminal variable domain (VL) is followed by a constant region consisting of a single domain termed CL.

The heavy and light chains pair by protein/protein interactions between the CH1 and CL domains, and via VH/VL interactions and the two heavy chains associate by protein/protein interactions between their CH3 domains. The structure of the immunoglobulin molecule is generally stabilized by interchains disulfide bonds between the CH1 and CL domains and between the hinges.

The antigen-binding regions or antigen-binding fragments correspond to the arms of the Y-shaped structure, which consist each of the complete light chain paired with the VH and CH1 domains of the heavy chain, and are called the Fab fragments (for Fragment antigen binding). Fab fragments were first generated from native immunoglobulin molecules by papain digestion which cleaves the antibody molecule in the hinge region, on the amino-terminal side of the interchains disulfide bonds, thus releasing two identical antigen-binding arms. Other proteases such as pepsin, also cleave the antibody molecule in the hinge region, but on the carboxy-terminal side of the interchains disulfide bonds, releasing fragments consisting of two identical Fab fragments and remaining linked through disulfide bonds; reduction of disulfide bonds in the F(ab′)2 fragments generates Fab′ fragments.

The part of the antigen binding region corresponding to the VH and VL domains is called the Fv fragment (for Fragment variable); it contains the CDRs (complementarity determining regions), which form the antigen-binding site (also termed paratope).

The effector region of the antibody which is responsible of its binding to effector molecules or cells, corresponds to the stem of the Y-shaped structure, and contains the paired CH2 and CH3 domains of the heavy chain (or the CH2, CH3 and CH4 domains, depending on the class of antibody), and is called the Fc (for Fragment crystallisable) region.

Due to the identity of the two heavy chains and the two light chains, naturally occurring antibody molecules have two identical antigen-binding sites and thus bind simultaneously to two identical epitopes.

An antibody “specifically binds” to a target antigen if it binds with greater affinity, avidity, more readily, and/or with greater duration than it binds to other substances. “Specific binding” or “preferential binding” does not necessarily require (although it can include) exclusive binding. Generally, but not necessarily, reference to binding means preferential binding.

The terms “subject,” “individual,” and “patient” are used interchangeably herein and refer to a mammal being assessed for treatment and/or being treated. Subjects may be human, but also include other mammals, particularly those mammals useful as laboratory models for human disease, e.g. mouse, rat, rabbit, dog, etc.

The term “treatment” or “treating” refers to an action, application or therapy, wherein a subject, including a human being, is subjected to medical aid with the purpose of improving the subject's condition, directly or indirectly. Particularly, the term refers to reducing incidence, or alleviating symptoms, eliminating recurrence, preventing recurrence, preventing incidence, improving symptoms, improving prognosis or combination thereof in some embodiments. The skilled artisan would understand that treatment does not necessarily result in the complete absence or removal of symptoms. For example, with respect to cancer, “treatment” or “treating” may refer to slowing neoplastic or malignant cell growth, proliferation, or metastasis, preventing or delaying the development of neoplastic or malignant cell growth, proliferation, or metastasis, or some combination thereof.

The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

The term “mutated derivative”, “mutant” or “functional derivative” designates a sequence that differs from the parent sequence to which it refers by deletion, substitution or insertion of one or several amino acids. Preferably, the mutated derivative has an amino acid sequence that is at least 80%, 85%, 90%, 91%, 92%, 93%, 94%, 95%, 96%, 97%, 98%, or 99% identical to the native sequence.

Cetuximab (Erbitux®; ImClone/Lilly, Merck-Serono) is a chimeric mouse-human monoclonal antibody (ATCC HB-9764 & ATCC-97-63) targeting epidermal growth factor receptor (EGFR). See also EP0359282, EP0667165, and U.S. Pat. No. 6,217,866. Cetuximab is approved for use as a treatment for colorectal cancer and squamous cell carcinoma of the head and neck.

Trastuzumab (Herceptin®; Genentech/Roche) is a humanized IgG1 that interferes with the HER2/neu receptor. See also EP0590058, U.S. Pat. Nos. 5,821,337, 8,075,890, 6,407,213, 6,054,297, 5,772,997, 6,165,464, 6,399,063 and 6,639,055. Its indications are the treatment of adjuvant and metastatic breast and metastatic gastric cancers.

Patritumab (U1-59/U3-1287/AMG888) is a fully human anti-HER3 monoclonal antibody with an IgG1-like isotype, possessing 1-3 nM affinity for its target, and is directed against the juxtamembrane portion of HER3. Patritumab has shown promising anti-tumor effects in vitro and in vivo in lung, head and neck, and breast cancer models where it effectively blocks HER3 phosphorylation, degrades the HER3 receptor, and reduces tumor burden.

Matuzumab (EMD72000) is a humanized IgG1 derived from the murine antibody MAb 425 (EMD55900) that was produced by lymphocyte hybridization from BALB/c mouse spleens immunized with human A431 squamous cell carcinoma cells. Matuzumab has been tested in Phase I clinical trials against a number of cancers, both alone and in combination with chemotherapy.

### 1. Design of Bispecific Antigen-Binding Fragments

The invention provides a bispecific bivalent antigen binding fragment, comprising one binding site to HER-3 and one binding site to another antigen selected from either HER-2 or EGFR antigens. In a particular embodiment, the bispecific antigen-binding fragment is capable of simultaneous binding to HER-3 and to HER-2. In another particular embodiment, the bispecific antigen-binding fragment is capable of simultaneous binding to HER-3 and to EGFR.

The antigen-binding fragment of the invention consists essentially of tandemly arranged Fab fragments. The invention relates specifically to bispecific antigen-binding fragments constructed using the amino acid sequences of the heavy chain (VH) and the light chain (VL) variable regions of two monoclonal antibodies “Ab1” and “Ab2”, wherein one of Ab1 or Ab2 is patritumab or a functional derivative thereof, and the other of Ab1 or Ab2 is selected from the group consisting of trastuzumab or a functional derivative thereof, matuzumab or a functional derivative thereof, and cetuximab or a functional derivative thereof.

Said bispecific antigen-binding fragment comprises:
(i) a Fab fragment comprising a VH-CH1 heavy chain associated with a VL-CL light chain of antibody 1 (Ab1), and
(ii) a Fab fragment comprising a VH-CH1 heavy chain associated with a VL-CL light chain of antibody 2 (Ab2),
   wherein the N-terminal end of the VH domain of the Fab fragment of Ab1 is linked to the C-terminal end of the CH1 domain of the Fab fragment of Ab2 through a polypeptide linker,
   wherein one of Ab1 or Ab2 is patritumab or a functional derivative thereof;
   and the other of Ab1 or Ab2 is selected from the group consisting of trastuzumab or a functional derivative thereof, matuzumab or a functional derivative thereof, and cetuximab or a functional derivative thereof.

In a particular embodiment, the bispecific antigen-binding fragment comprises two Fab fragments with different CH1 and CL domains. In a preferred embodiment, the bispecific antigen-binding fragment comprises two Fab fragments with different CH1 and CL domains consisting of:
(i) a Fab fragment having CH1 and CL domains derived from a human IgG1/Kappa, and the VH and VL domains of Ab1,
(ii) a Fab fragment having CH1 and CL domains derived from a human IgG1/Kappa and the VH and VL domains of Ab2.

In a particular embodiment, Ab1 is patritumab or a functional derivative thereof and Ab2 is trastuzumab or a functional derivative thereof. In another particular embodiment, Ab1 is trastuzumab or a functional derivative thereof and Ab2 is patritumab or a functional derivative thereof.

In a particular embodiment, Ab1 is patritumab or a functional derivative thereof and Ab2 is cetuximab or a functional derivative thereof. In another particular embodiment, Ab1 is cetuximab or a functional derivative thereof and Ab2 is patritumab or a functional derivative thereof.

In a particular embodiment, Ab1 is patritumab or a functional derivative thereof and Ab2 is matuzumab or a functional derivative thereof. In another particular embodiment, Ab1 is matuzumab or a functional derivative thereof and Ab2 is patritumab or a functional derivative thereof.

In a preferred embodiment, Ab2 is patritumab or a functional derivative thereof and Ab1 is selected from the group consisting of trastuzumab or a functional derivative thereof, matuzumab or a functional derivative thereof, and cetuximab or a functional derivative thereof.

In a particular embodiment, the bispecific antigen-binding fragment comprises a Fab fragment comprising a VH-CH1 heavy chain associated with a VL-CL light chain of patritumab or a functional derivative thereof, wherein:
the VH domain comprises, preferably consists of SEQ ID NO: 5, or a mutated derivative thereof; and
the VL domain comprises, preferably consists of SEQ ID NO: 6, or a mutated derivative thereof;
   wherein, the CH1 domain is preferably a CH1 constant domain of human IgG1 or a mutated derivative thereof;
   and wherein the CL domain is preferably a human Kappa constant domain or a mutated derivative thereof.

In a particular embodiment, the bispecific antigen-binding fragment comprises a Fab fragment comprising a VH-CH1 heavy chain associated with a VL-CL light chain of cetuximab or a functional derivative thereof, wherein:
the VH domain comprises, preferably consists of SEQ ID NO: 7, or a mutated derivative thereof; and
the VL domain comprises, preferably consists of SEQ ID NO: 8, or a mutated derivative thereof;
   wherein, the CH1 domain is preferably a CH1 constant domain of human IgG1 or a mutated derivative thereof;
   and wherein the CL domain is preferably a human Kappa constant domain or a mutated derivative thereof.

In a particular embodiment, the bispecific antigen-binding fragment comprises a Fab fragment comprising a VH-CH1 heavy chain associated with a VL-CL light chain of trastuzumab or a functional derivative thereof, wherein:
the VH domain comprises, preferably consists of SEQ ID NO: 9, or a mutated derivative thereof; and
the VL domain comprises, preferably consists of SEQ ID NO: 10, or a mutated derivative thereof;
   wherein, the CH1 domain is preferably a CH1 constant domain of human IgG1 or a mutated derivative thereof;
   and wherein the CL domain is preferably a human Kappa constant domain or a mutated derivative thereof.

In a particular embodiment, the bispecific antigen-binding fragment comprises a Fab fragment comprising a VH-CH1 heavy chain associated with a VL-CL light chain of matuzumab or a functional derivative thereof, wherein:
the VH domain comprises, preferably consists of SEQ ID NO: 11, or a mutated derivative thereof; and
the VL domain comprises, preferably consists of SEQ ID NO: 12, or a mutated derivative thereof;
   wherein, the CH1 domain is preferably a CH1 constant domain of human IgG1 or a mutated derivative thereof;
   and wherein the CL domain is preferably a human Kappa constant domain or a mutated derivative thereof.

### Mutated Derivatives

The invention makes use of wild-type sequences (of patritumab, matuzumab, cetuximab or trastuzumab), or mutated derivates thereof.

The term “mutated derivative”, “mutant”, or “functional derivative” designates a sequence that differs from the parent sequence to which it refers by deletion, substitution or insertion of one or several amino acids. Preferably, the mutated derivative or functional variant has an amino acid sequence that is at least 80%, 85%, 90%, 91%, 92%, 93%, 94%, 95%, 96%, 97%, 98%, or 99% identical to the native sequence. In a particular embodiment, the mutations do not substantially impact the function of the antibody.

Mutated derivatives, or functional variants, can comprise a VH chain that comprises an amino acid sequence at least 80% (e.g., at least 85%, 90%, 91%, 92%, 93%, 94%, 95%, 96%, 97%, 98%, or 99%) identical to any of the reference sequences recited herein, a VL chain that has an amino acid sequence at least 80% (e.g., at least 85%, 90%, 91% 92%, 93%, 94%, 95%, 96%, 97%, 98%, or 99%) identical to any of the reference sequences recited herein, or both. The functional variant of patritumab is capable of binding to HER3. The functional variant of cetuximab is capable of binding to EGFR. The functional variant of matuzumab is capable of binding to EGFR. The functional variant of trastuzumab is capable of binding to HER2.

In some examples, the variants possess similar antigen-binding affinity relative to the reference antibodies described above (e.g., having a Kd less than 1×10⁻⁸, preferably less than 1×10⁻⁹ or 1×10⁻¹⁰ M). In some embodiments, the functional variant of the original antibody has the same binding specificity and has an affinity with its target that is at least 50%, such as at least 60%, 70%, 80% or at least 90% of the affinity of the original antibody.

The affinity of the binding is defined by the terms ka (associate rate constant), kd (dissociation rate constant), or KD (equilibrium dissociation). Typically, specifically binding when used with respect to an antibody refers to an antibody that specifically binds to (“recognizes”) its target(s) with an affinity (KD) value less than 10⁻⁸ M, e.g., less than 10⁻⁹ M or 10⁻¹⁰ M. A lower KD value represents a higher binding affinity (i.e., stronger binding) so that a KD value of 10⁻⁹ indicates a higher binding affinity than a KD value of 10⁻⁸.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25 (17): 3389-3402, 1997. When utilizing BLAST and Gapped BLAST programs, the default parameters of the respective programs (e.g., XBLAST and NBLAST) can be used.

In other embodiments, the functional variants described herein can contain one or more mutations (e.g., conservative substitutions) which preferably do not occur at residues which are predicted to interact with one or more of the CDRs.

It is herein described mutated derivatives, or functional variants, which are substantially identical to the reference antibody.

The term “substantially identical” or “insubstantial” means that the relevant amino acid sequences (e.g., in framework regions (FRs), CDRs, VH, or VL domain) of a variant differ insubstantially (e.g., including conservative amino acid substitutions) as compared with a reference antibody such that the variant has substantially similar binding activities (e.g., affinity, specificity, or both) and bioactivities relative to the reference antibody. Such a variant may include minor amino acid changes, e.g. 1 or 2 substitutions in a 5 amino acid sequence of a specified region. Generally, more substitutions can be made in FR regions, in contrast to CDR regions, as long as they do not adversely impact the binding function of the antibody (such as reducing the binding affinity by more than 50% as compared to the original antibody). In some embodiment, the sequence identity can be about 85%, 90%, 95%, 96%, 97%, 98%, 99% or higher, between the original and the modified antibody. In some embodiments, the modified antibody has the same binding specificity and has at least 50% of the affinity of the original antibody. In some embodiments, the mutations do not occur within the CDR regions.

Conservative substitutions will produce molecules having functional and chemical characteristics similar to those of the molecule from which such modifications are made. For example, a “conservative amino acid substitution” may involve a substitution of a native amino acid residue with another residue such that there is little or no effect on the polarity or charge of the amino acid residue at that position. Desired amino acid substitutions (whether conservative or non-conservative) can be determined by those skilled in the art. For example, amino acid substitutions can be used to identify important residues of the molecule sequence, or to increase or decrease the affinity of the molecules described herein. Variants comprising one or more conservative amino acid substitutions can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

The present disclosure also provides antibody variants with improved biological properties of the antibody, such as higher or lower binding affinity, or with altered ADCC properties, or with altered effects of viability inhibition of HER3, EGFR and/or HER2 expressing cells.

Amino acid sequence variants of the antibody can be prepared by introducing appropriate nucleotide changes into the antibody nucleic acid, or via peptide synthesis. Such modifications include, for example, deletions from, and/or insertions into and/or substitutions of, residues within the amino acid sequences of the antibody. Any combination of deletion, insertion, and substitution is made to achieve the final construct, provided that the final construct possesses the desired characteristics. Nucleic acid molecules encoding amino acid sequence variants of the antibody can be prepared by a variety of methods known in the art. These methods include, but are not limited to, oligonucleotide-mediated (or site-directed) mutagenesis, PCR mutagenesis, and cassette mutagenesis of an earlier prepared variant or a non-variant (natural) version of the antibody. In one embodiment, the equilibrium dissociation constant (KD) value of the antibodies of the invention is less than 10⁻⁸ M, particularly less than 10⁻⁹ M or 10⁻¹⁰ M. The binding affinity may be determined using techniques known in the art, such as ELISA or biospecific interaction analysis, or other techniques known in the art.

Any of the antibodies described herein can be examined to determine their properties, such as antigen-binding activity, antigen-binding specificity, and biological functions, following routine methods.

Any of the antibodies described herein can be modified to contain additional nonproteinaceous moieties that are known in the art and readily available, e.g., by PEGylation, hyperglycosylation, conjugation of toxins, radioactive labels and the like. Modifications that can enhance serum half-life are of interest.

The antibodies of the invention may be glycosylated or not, or may show a variety of glycosylation profiles. In a preferred embodiment, antibodies are unglycosylated on the variable region of the heavy and light chains, but are glycosylated on the Fc region.

For example to remove the N-glycosylation site in the VH domain of cetuximab, the Asn at Kabat position H85 is mutated to aspartic acid (D) according to the sequence SEQ ID NO: 77, or the Asn at Kabat position H85 is mutated to glutamic acid (E) according to the sequence SEQ ID NO: 78.

Certain mutated derivatives of cetuximab are humanized forms of the reference cetuximab antibody, which, in its original form, is a chimeric antibody with heavy and light chain variable regions of murine origin. In a humanization approach, complementarity determining regions (CDRs) and certain other amino acids from donor mouse variable regions are grafted into human variable acceptor regions and then joined to human constant regions; some CDR amino acids may be further replaced by amino acids found in human germline sequences of the acceptor regions. See, e.g. Riechmann et al., Nature 332:323-327 (1988); U.S. Pat. No. 5,225,539.

The invention further encompasses bispecific antigen-binding fragment containing humanized version of light chains and/or heavy chains of cetuximab.

In a particular embodiment, Ab1 or Ab2 is cetuximab or a humanized version of cetuximab. In a particular embodiment, the humanized form of cetuximab has a VH domain having an amino acid sequence which is at least 80%, 85%, 90%, 92%, 94%, 96%, 98%, or at least 99% identical to SEQ ID NO: 7; and a VL domain having an amino acid sequence which is at least 80%, 85%, 90%, 92%, 94%, 96%, 98%, or at least 99% identical to SEQ ID NO: 8.

In a particular embodiment, the humanized form of cetuximab is any humanized form described in patent application WO2012020059.

In a particular embodiment, Ab1 or Ab2 is a humanized form of cetuximab which is capable of binding to EGFR and which comprises:
a heavy chain variable region (VH) comprising a CDR1 having the amino acid sequence of SEQ ID NO: 49, a CDR2 having the amino acid sequence of SEQ ID NO: 50, and a CDR3 having the amino acid sequence of SEQ ID NO: 51; and/or
a light chain variable region (VL) comprising a CDR1 having the amino acid sequence of SEQ ID NO: 52, a CDR2 having the amino acid sequence of SEQ ID NO: 53, and a CDR3 having the amino acid sequence of SEQ ID NO: 54.

In one embodiment, at least 2 and preferably all of the framework regions 1, 2 and 3 of the heavy chain variable region (VH) of the humanized cetuximab are derived from or correspond to the human germline VH gene 4-59*01 coding for an amino acid sequence comprising SEQ ID NO: 55. Furthermore, the framework region 4 of the heavy chain variable region (VH) is preferably derived from or corresponds to the human germline gene JH1 coding for an amino acid sequence comprising SEQ ID NO: 56. In one embodiment, at least one of framework regions 1, 2 and 3 of the light chain variable region (VL) of the humanized cetuximab is derived from or corresponds to the human germline VL gene 6-21 *01 coding for an amino acid sequence comprising SEQ ID NO: 57. Preferably, at least 2, more preferably all 3 of framework regions 1, 2 and 3 of the light chain variable region are derived from or correspond to the human germline VL gene 6-21*01 coding for an amino acid sequence comprising SEQ ID NO: 57. Furthermore, the framework region 4 of the light chain variable region (VL) is preferably derived from or corresponds to the human germline gene JK2 coding for an amino acid sequence comprising SEQ ID NO: 58.

Preferably, the humanized cetuximab comprises a heavy chain variable region (VH) comprising the amino acid sequence of SEQ ID NO: 59 and/or a light chain variable region (VL) comprising the amino acid sequence of SEQ ID NO: 60. The heavy chain variable region (VH) preferably comprises an amino acid sequence selected from the group consisting of SEQ ID NOs: 61 to 68, in particular the amino acid sequence of SEQ ID NO: 63. In a particular embodiment, the heavy chain variable region (VH) of humanized cetuximab preferably comprises the amino acid sequence of SEQ ID NO: 81. Furthermore, the light chain variable region (VL) preferably comprises an amino acid sequence selected from the group consisting of SEQ ID NOs: 69 to 72, in particular the amino acid sequence of SEQ ID NO: 71. Particularly preferred is a humanized antibody comprising a heavy chain variable region (VH) comprising the amino acid sequence of SEQ ID NO: 63 and a light chain variable region (VL) comprising the amino acid sequence of SEQ ID NO: 71. Another particularly preferred is a humanized cetuximab comprising a heavy chain variable region (VH) comprising the amino acid sequence of SEQ ID NO: 81 and a light chain variable region (VL) comprising the amino acid sequence of SEQ ID NO: 71.

In a particular embodiment, the humanized Cetuximab comprises:
a VH domain of SEQ ID NO: 81 and a CH1 domain of SEQ ID NO: 2 (corresponding to VH-CH1 sequence of SEQ ID NO: 83)
a VL domain of SEQ ID NO: 71 and a CL domain of SEQ ID NO: 4 (corresponding to VL-CL sequence of SEQ ID NO: 84).

In a particular embodiment, the humanized cetuximab comprises a heavy chain variable region (VH) comprising an amino acid sequence having at least 80%, 85%, 90%, 92%, 94%, 96%, 98%, or at least 99% identity with SEQ ID NO: 59 or with SEQ ID NOs: 61 to 68, in particular with the amino acid sequence of SEQ ID NO: 63. In a particular embodiment, the humanized cetuximab comprises a light chain variable region (VL) comprising an amino acid sequence having at least 80%, 85%, 90%, 92%, 94%, 96%, 98%, or at least 99% identity with SEQ ID NO: 60 or with SEQ ID NOs: 69 to 72, in particular with the amino acid sequence of SEQ ID NO: 71.

In some examples, Ab1 or Ab2 is a humanized cetuximab which comprises:
a Light chain humanized version of cetuximab based on human immunoglobulin gene kappa variable 6-11 allele 02 (IGKV6-21*02) as defined in IMGT/Gene database,

(SEQ ID NO: 73)
EIVLTQSPDFQSVTPKEKVTITCRASQSIGTNIHWYQQKPDQSPKLLIK
YASESISGVPSRFSGSGSGTDFTLTINSLEAEDAATYYCQQNNNWPTTF
GQGTKLEIK

Where at the following Kabat positions the amino acid residues are:
Kabat position L31 a Thr or Ser
Kabat position L32 an Asn or Ser
Kabat position L33 a Ile or Leu
Kabat position L53 a Glu or GinKabat position L89 a Gln or His
And optionally, Kabat position L91 an Asn, Ser, His, Lys or Arg
Kabat position L92 an Asn, Ser, His, Lys or Arg
Kabat position L93 an Asn, Ser, His, Lys or Arg
Kabat position L94 a Trp, Tyr or Phe
Kabat position L96 a Thr or Tyr.

For clarification purposes, L31 refers to the residues 31 in the Light Chain.
a Light chain humanized version of cetuximab based on human immunoglobulin gene kappa variable 3-11 allele 01 (IGKV3-11*01) as defined in IMGT/Gene database,

(SEQ ID NO: 74)
EIVLTQSPATLSLSPGERATLSCRASQSIGTNIHWYQQKPGQAPRLLIK
YASESISGIPARFSGSGSGTDFTLTISSLEPEDFAVYYCQQNNNWPTTF
GQGTKLEIK

Where at the following Kabat positions the amino acid residues are:
Kabat position L29 an Ile or Val
Kabat position L30 a Gly or Ser
Kabat position L31 a Thr or Ser
Kabat position L32 an Asn or Tyr
Kabat position L33 a Ile or Leu
Kabat position L34 a His or Ala
Kabat position L49 a Lys or Tyr
Kabat position L50 a Tyr or Asp
Kabat position L53 a Glu or Asn
Kabat position L54 a Ser or Arg
Kabat position L55 an Ile or Ala
Kabat position L56 a Ser or Thr
And optionally Kabat position L91 an Asn, Arg, His, or Lys
Kabat position L92 an Asn, Ser, His, Lys or Arg
Kabat position L94 a Trp, Tyr or Phe
Kabat position L96 a Thr or Tyr.
   a Heavy chain humanized version of cetuximab mAb based on human immunoglobulin gene heavy variable 4-59 allele 01 (IGHV4-59*01) as defined in IMGT/Gene database,

(SEQ ID NO: 75)
QVQLQESGPGLVKPSETLSLTCTVSGFSLTNYGVHWVRQPPGKGLEWLG
VIWSGGNTDYNTPLTSRLTISKDNSKNQVSLKLSSVTAADTAVYYCARA
LTYYDYEFAYWGQGTLVTVSS

Where at the following Kabat positions the amino acid residues are:
Kabat position H29 a Leu or Ile
Kabat position H30 a Thr or Ser
Kabat position H31 an Asn or Ser
Kabat position H33 a Gly or Tyr
Kabat position H35 a His or Ser
Kabat position H37 a Val or Ile
Kabat position H48 a Leu or Ile
Kabat position H50 at Val or Tyr
Kabat position H52 a Trp, Tyr or Phe
Kabat position H53 a Ser or Tyr
Kabat position H54 a Gly or Ser
Kabat position H56 an Asn or Ser
Kabat position H58 a Asp or Asn
Kabat position H61 a Thr or Pro
Kabat position H62 a Pro or Ser
Kabat position H64 a Thr or Lys
Kabat position H67 a Leu or Val
Kabat position H73 a Asn or Thr
Kabat position H78 a Val or Phe.

For clarification purposes, H29 refers to the residue 29 in the Heavy Chain.
a Heavy chain humanized version of cetuximab mAb based on human immunoglobulin gene heavy variable 3-33 allele 01 (IGHV3-33*01) as defined in IMGT/Gene database,

(SEQ ID NO: 76)
QVQLVESGGGVVQPGRSLRLSCAVSGFSLTNYGVHWVRQAPGKGLEWLG
VIWSGGNTDYNTPVTSRFTISKDNSKNTVYLQMNSLRAEDTAVYYCARA
LTYYDYEFAYWGQGTLVTVSS

Where at the following Kabat positions the amino acid residues are:
Kabat position H28 a Ser or Thr
Kabat position H30 a Thr or Ser
Kabat position H48 a Leu or Val
Kabat position H49 a Gly or Ala
Kabat position H53 a Ser or Asp
Kabat position H55 a Gly or Ser
Kabat position H57 a Lys or Thr
Kabat position H58 a Asp or Tyr
Kabat position H60 an Asn or Ala
Kabat position H61 a Thr or Asp
Kabat position H62 a Pro or Ser
Kabat position H64 a Thr or Lys
Kabat position H65 a Ser or Gly
Kabat position H78 a Val or Leu.

In a particular embodiment, the Fab fragment of Ab1 and/or the Fab fragment of Ab2 comprises mutations in the VH/VL domains to facilitate pairing of the Fab light chain with the Fab heavy chain. Specific mutations are described in international patent application WO2020/136566, all incorporated herein by reference.

In a particular embodiment, the Fab fragment of Ab1 and/or the Fab fragment of Ab2 comprises a mutated VH domain wherein residue at Kabat position 39 has been mutated from Glutamine to Lysine; and a VL domain wherein residue at Kabat position 38 has been mutated from Glutamine to Glutamic acid.

In a particular embodiment, the Fab fragment of Ab1 and/or the Fab fragment of Ab2 comprises a mutated VH domain wherein residue at Kabat position 39 has been mutated from Glutamine to Lysine; and a VL domain wherein residue at Kabat position 38 has been mutated from Glutamine to Aspartic acid.

In a particular embodiment, the Fab fragment of Ab1 and/or the Fab fragment of Ab2 comprises a mutated VH domain wherein residue at Kabat position 39 has been mutated from Glutamine to Glutamic acid; and a VL domain wherein residue at Kabat position 38 has been mutated from Glutamine to Lysine.

### CH1/CL Mutated Domains

The assembly of Fab domains is accomplished via natural pairing of Light and Heavy chains without the use of peptide linkers. In order to maximize propensity of cognate pairing between Light and Heavy chains, one may contemplate introducing mutations at the interface of Light and Heavy chains (CL/CH1 interface) in Fab fragments.

In a particular embodiment, the CH1 domain of Ab1 and/or Ab2 is a CH1 constant domain from human IgG1 of any allotype, or a mutated derivative thereof. In a particular embodiment, the CH1 domain of Ab1 and/or Ab2 is a CH1 domain of human IgG1 of G1m (1,17) allotype or a mutated derivative thereof. In another particular embodiment, the CH1 domain of Ab1 and/or Ab2 is a CH1 domain of human IgG1 of G1m (3) allotype or a mutated derivative thereof.

In preferred embodiments, each CH1 domain carries at least one mutation, and each CL1 domain also carries at least one mutation, which mutations are selected so that a correct cognate pairing of the CH1 and CL1 domains is improved.

These mutations can be selected from the following list:
de novo-introduced ionic pairs or reversed polarity charged mutations of native ionic pairs already present at the interface of the Heavy and Light chains of the Fab fragment;
knobs-into-holes” mutations;
mutations that resurface opposing constant regions of Heavy and Light chain interfaces in Fab fragments to change them from strongly polar to highly hydrophobic or vice versa.

Several sets of mutations are thus suitable, as described in greater details below.

Of note, throughout the present description, amino acid sequences and the sequence position numbers used herein for the CH1 and CL domains are defined according to Kabat et al, Sequences of Proteins of Immunological Interest, 5th Ed. Public Health Service, National Institutes of Health, Bethesda, Md. (1991).

Residues that can be mutated in the CL kappa domain may be e.g. selected from the group consisting of S 114, F 116, F 118, E 123 (e.g. E123K), Q 124, T 129, S 131, V 133, L 135, N 137, Q 160, S 162, S 174, S 176, T 178, and T 180.

Residues that can be mutated in the CH1 domain may be e.g. selected from the group consisting of L 124, A 139, L 143, D 144, K 145, D 146, H 172, F 174, P 175, Q 179, S183, S 188, V 190, T 192, and K 221 (e.g. K221E).

Specific mutations are described in US patent applications 2014/0200331, 2014/150973, 2014/0154254, and international patent application WO2007/147901, all incorporated herein by reference.

Examples of CH1/CL mutated derivatives are described below.

According to a particular embodiment, the bispecific antigen-binding fragment comprises:
a mutated CH1 constant domain from human IgG1 (residue at Kabat position 192 has been mutated from threonine to Glutamic acid (E)) corresponding to SEQ ID NO: 1;
a mutated CH1 constant domain from human IgG1 (residue at Kabat position 192 has been mutated from threonine to aspartic acid (D)) corresponding to SEQ ID NO: 45;
a mutated CH1 constant domain from human IgG1 (residue at Kabat position 143 has been mutated from leucine to Glutamine (Q); and residue at Kabat position 188 has been mutated from serine to valine (V)) corresponding to SEQ ID NO: 2;
a mutated CH1 constant domain from human IgG1 (residue at Kabat position 124 has been mutated from leucine to glutamine (Q); and residue at Kabat position 188 has been mutated from serine to valine (V)) corresponding to SEQ ID NO: 46;
a mutated human Kappa constant domain (residue at Kabat positions 114 has been mutated from serine to alanine (A) and residue at Kabat position 137 has been mutated from asparagine to lysine (K)) corresponding to SEQ ID NO: 3;
a mutated human Kappa constant domain (residue at Kabat positions 133 has been mutated from valine to threonine (T) and residue at Kabat position 176 has been mutated from serine to valine (V)) corresponding to SEQ ID NO: 4.

In a preferred embodiment, a pair of interacting polar interface residues is exchanged for a pair of neutral and salt bridge forming residues. The replacement of Thr192 by a glutamic acid or aspartic acid on CH1 chain and exchange of Asn137 to a Lys on CL chain can be selected, optionally with a substitution of the serine residue at position 114 of said CL domain with an alanine residue.

In another set of mutations, one can replace the Leu143 of the CH1 domain by a Gln residue, while the facing residue of the CL chain, that is Val33, is replaced by a Thr residue. This first double mutation constitutes the switch from hydrophobic to polar interactions. Simultaneously a mutation of two interacting serines (Ser188 on CH1 chain and Ser176 on CL chain) to valine residues can achieve a switch from polar to hydrophobic interactions.

In yet another embodiment, the mutations can comprise substitution of the leucine residue at position 124 of CH1 domain with a glutamine and substitution of the serine residue at position 188 of CH1 domain with a valine residue; and substitution of the valine residue at position 133 of CL domain with a threonine residue and substitution of the serine residue at position 176 of said CL domain with a valine residue.

The “knob into holes” mutations include a set of mutations (KH1) wherein Leu 124 and Leu 143 of the CH1 domain have been respectively replaced by an Ala and a Glu residue while the Val 33 of the CL chain has been replaced by a Trp residue, while, in the set of mutations named H2, the Val 90 of the CH1 domain has been replaced by an Ala residue, and the Leu135 and Asn137 of the CL chain have respectively been replaced by a Trp and an Ala residue.

The preferred mutations are disclosed in the table below:

| Preferred | |
|---|---|
| mutations | |
| (LC is Light Chain, HC is | Name of the |
| Heavy Chain) | mutation set |
| LC(S114A/N137K), HC(T192E) | CR3 mutation |
| LC(S114A/N137K), HC(T192D) | CC1 mutation |
| LC(V133T/S176V), | Mut4 |
| HC(L143Q/S188V) | |
| LC(V133T/S176V), | ML1 |
| HC(L124Q/S188V) | |

In a particular embodiment, the multispecific antibody may carry a double mutation, e.g. one Fab with the CR3 mutation and the other Fab with the Mut4 mutation.

Bispecific antigen-binding fragment are more particularly described, wherein Ab1 or Ab2 is patritumab or a mutated derivative thereof, comprising:
a VH domain consisting of SEQ ID NO: 5 or a mutated derivative thereof,
a CH1 domain selected from the group consisting of SEQ ID NO: 1, SEQ ID NO: 2, SEQ ID NO: 45, SEQ ID NO: 46 and SEQ ID NO: 47,
a VL domain consisting of SEQ ID NO: 6 or a mutated derivative thereof,
a CL domain selected from the group consisting of SEQ ID NO: 3, SEQ ID NO: 4 and SEQ ID NO: 48,
   wherein the CL and CH1 domains preferably associate as follows:
SEQ ID NO: 48 with SEQ ID NO: 47,
SEQ ID NO: 3 with either SEQ ID NO: 1 or SEQ ID NO: 45,
SEQ ID NO: 4 with either SEQ ID NO: 2 or SEQ ID NO: 46.

Preferably, Ab1 or Ab2 is patritumab or a mutated derivative thereof, comprising:
a VH domain consisting of SEQ ID NO: 5 or a mutated derivative thereof,
a CH1 domain consisting of SEQ ID NO: 1 or a mutated derivative thereof,
a VL domain consisting of SEQ ID NO: 6 or a mutated derivative thereof,
a CL domain consisting of SEQ ID NO: 3 or a mutated derivative thereof.

Preferably, Ab1 or Ab2 is patritumab or a mutated derivative thereof, comprising:
a VH-CH1 heavy chain comprising or consisting of SEQ ID NO: 13 or a functional variant having at least 80%, 85%, 90%, 92%, 94%, 96%, 98% or at least 99% sequence identity with SEQ ID NO: 13; and
a VL-CL light chain comprising or consisting of SEQ ID NO: 14, or a functional variant having at least 80%, 85%, 90%, 92%, 94%, 96%, 98% or at least 99% sequence identity with SEQ ID NO: 14.

Bispecific antigen-binding fragment are more particularly described, wherein Ab1 or Ab2 is cetuximab or a mutated derivative thereof, comprising:
a VH domain consisting of SEQ ID NO: 7, a mutated derivative thereof or a humanized derivative thereof,
a CH1 domain selected from the group consisting of SEQ ID NO: 1, SEQ ID NO: 2, SEQ ID NO: 45, SEQ ID NO: 46 and SEQ ID NO: 47,
a VL domain consisting of SEQ ID NO: 8, a mutated derivative thereof or a humanized derivative thereof,
a CL domain selected from the group consisting of SEQ ID NO: 3, SEQ ID NO: 4 and SEQ ID NO: 48,
   wherein the CL and CH1 domains preferably associate as follows:
SEQ ID NO: 48 with SEQ ID NO: 47,
SEQ ID NO: 3 with either SEQ ID NO: 1 or SEQ ID NO: 45,
SEQ ID NO: 4 with either SEQ ID NO: 2 or SEQ ID NO: 46.

Preferably, Ab1 or Ab2 is cetuximab or a mutated derivative thereof, comprising:
a VH domain consisting of SEQ ID NO: 7, a mutated derivative thereof or a humanized derivative thereof,
a CH1 domain consisting of SEQ ID NO: 2 or a mutated derivative thereof,
a VL domain consisting of SEQ ID NO: 8, a mutated derivative thereof or a humanized derivative thereof,
a CL domain consisting of SEQ ID NO: 4 or a mutated derivative thereof.

Preferably, Ab1 or Ab2 is cetuximab or a mutated derivative thereof, comprising:
a VH-CH1 heavy chain comprising or consisting of SEQ ID NO: 15 or a functional variant having at least 80%, 85%, 90%, 92%, 94%, 96%, 98% or at least 99% sequence identity with SEQ ID NO: 15; and
a VL-CL light chain comprising or consisting of SEQ ID NO: 16, or a functional variant having at least 80%, 85%, 90%, 92%, 94%, 96%, 98% or at least 99% sequence identity with SEQ ID NO: 16.

Bispecific antigen-binding fragment are more particularly described, wherein Ab1 or Ab2 is trastuzumab or a mutated derivative thereof, comprising:
a VH domain consisting of SEQ ID NO: 9 or a mutated derivative thereof,
a CH1 domain selected from the group consisting of SEQ ID NO: 1, SEQ ID NO: 2, SEQ ID NO: 45, SEQ ID NO: 46 and SEQ ID NO: 47,
a VL domain consisting of SEQ ID NO: 10 or a mutated derivative thereof,
a CL domain selected from the group consisting of SEQ ID NO: 3, SEQ ID NO: 4 and SEQ ID NO: 48,
   wherein the CL and CH1 domains preferably associate as follows:
SEQ ID NO: 48 with SEQ ID NO: 47,
SEQ ID NO: 3 with either SEQ ID NO: 1 or SEQ ID NO: 45,
SEQ ID NO: 4 with either SEQ ID NO: 2 or SEQ ID NO: 46.

Preferably, Ab1 or Ab2 is trastuzumab or a mutated derivative thereof, comprising:
a VH domain consisting of SEQ ID NO: 9 or a mutated derivative thereof,
a CH1 domain consisting of SEQ ID NO: 2 or a mutated derivative thereof,
a VL domain consisting of SEQ ID NO: 10 or a mutated derivative thereof,
a CL domain consisting of SEQ ID NO: 4 or a mutated derivative thereof.

Preferably, Ab1 or Ab2 is trastuzumab or a mutated derivative thereof, comprising:
a VH-CH1 heavy chain comprising or consisting of SEQ ID NO: 17 or a functional variant having at least 80%, 85%, 90%, 92%, 94%, 96%, 98% or at least 99% sequence identity with SEQ ID NO: 17; and
a VL-CL light chain comprising or consisting of SEQ ID NO: 18, or a functional variant having at least 80%, 85%, 90%, 92%, 94%, 96%, 98% or at least 99% sequence identity with SEQ ID NO: 18.

Bispecific antigen-binding fragment are more particularly described, wherein Ab1 or Ab2 is matuzumab or a mutated derivative thereof, comprising:
a VH domain consisting of SEQ ID NO: 11 or a mutated derivative thereof,
a CH1 domain selected from the group consisting of SEQ ID NO: 1, SEQ ID NO: 2, SEQ ID NO: 45, SEQ ID NO: 46 and SEQ ID NO: 47,
a VL domain consisting of SEQ ID NO: 12 or a mutated derivative thereof,
a CL domain selected from the group consisting of SEQ ID NO: 3, SEQ ID NO: 4 and SEQ ID NO: 48,
   wherein the CL and CH1 domains preferably associate as follows:
SEQ ID NO: 48 with SEQ ID NO: 47,
SEQ ID NO: 3 with either SEQ ID NO: 1 or SEQ ID NO: 45,
SEQ ID NO: 4 with either SEQ ID NO: 2 or SEQ ID NO: 46.

Preferably, Ab1 or Ab2 is matuzumab or a mutated derivative thereof, comprising:
a VH domain consisting of SEQ ID NO: 11 or a mutated derivative thereof,
a CH1 domain consisting of SEQ ID NO: 2 or a mutated derivative thereof,
a VL domain consisting of SEQ ID NO: 12 or a mutated derivative thereof,
a CL domain consisting of SEQ ID NO: 4 or a mutated derivative thereof.

Preferably, Ab1 or Ab2 is matuzumab or a mutated derivative thereof, comprising:
a VH-CH1 heavy chain comprising or consisting of SEQ ID NO: 19 or a functional variant having at least 80%, 85%, 90%, 92%, 94%, 96%, 98% or at least 99% sequence identity with SEQ ID NO: 19; and
a VL-CL light chain comprising or consisting of SEQ ID NO: 20, or a functional variant having at least 80%, 85%, 90%, 92%, 94%, 96%, 98% or at least 99% sequence identity with SEQ ID NO: 20.

In a particular embodiment, the bispecific antigen-binding fragment comprises:
(i) a Fab fragment comprising a VH-CH1 heavy chain associated with a VL-CL light chain of trastuzumab or a functional derivative thereof, and
(ii) a Fab fragment comprising a VH-CH1 heavy chain associated with a VL-CL light chain of patritumab or a functional derivative thereof,
wherein the N-terminal end of the VH domain of the Fab fragment of trastuzumab is linked to the C-terminal end of the CH1 domain of the Fab fragment of patritumab through a polypeptide linker,
wherein the VH-CH1 heavy chain of trastuzumab preferably consists of SEQ ID NO: 17, the VL-CL light chain of trastuzumab preferably consists of SEQ ID NO: 18, the VH-CH1 heavy chain of patritumab preferably consists of SEQ ID NO: 13, and/or wherein the VL-CL light chain of patritumab preferably consists of SEQ ID NO: 14.

In a preferred embodiment, the bispecific antigen binding fragment comprises one heavy chain comprising or consisting of SEQ ID NO:21 or a functional variant thereof, and two different light chains comprising or consisting of SEQ ID NO: 18 and SEQ ID NO: 14 or functional variants thereof.

In a particular embodiment, the bispecific antigen-binding fragment comprises:
(i) a Fab fragment comprising a VH-CH1 heavy chain associated with a VL-CL light chain of patritumab or a functional derivative thereof, and
(ii) a Fab fragment comprising a VH-CH1 heavy chain associated with a VL-CL light chain of trastuzumab or a functional derivative thereof,
wherein the N-terminal end of the VH domain of the Fab fragment of patritumab is linked to the C-terminal end of the CH1 domain of the Fab fragment of trastuzumab through a polypeptide linker,
wherein the VH-CH1 heavy chain of trastuzumab preferably consists of SEQ ID NO: 17, the VL-CL light chain of trastuzumab preferably consists of SEQ ID NO: 18, the VH-CH1 heavy chain of patritumab preferably consists of SEQ ID NO: 13, and/or wherein the VL-CL light chain of patritumab preferably consists of SEQ ID NO: 14.

In a preferred embodiment, the bispecific antigen binding fragment comprises one heavy chain comprising or consisting of SEQ ID NO: 22 or a functional variant thereof, and two different light chains comprising or consisting of SEQ ID NO: 18 and SEQ ID NO: 14 or functional variants thereof.

In a particular embodiment, the bispecific antigen-binding fragment comprises:
(i) a Fab fragment comprising a VH-CH1 heavy chain associated with a VL-CL light chain of cetuximab or a functional or humanized derivative thereof, and
(ii) a Fab fragment comprising a VH-CH1 heavy chain associated with a VL-CL light chain of patritumab or a functional derivative thereof,
wherein the N-terminal end of the VH domain of the Fab fragment of cetuximab is linked to the C-terminal end of the CH1 domain of the Fab fragment of patritumab through a polypeptide linker,
wherein the VH-CH1 heavy chain of cetuximab preferably consists of SEQ ID NO: 15, the VL-CL light chain of cetuximab preferably consists of SEQ ID NO: 16, the VH-CH1 heavy chain of patritumab preferably consists of SEQ ID NO: 13, and/or wherein the VL-CL light chain of patritumab preferably consists of SEQ ID NO: 14.

In a preferred embodiment, the bispecific antigen binding fragment comprises one heavy chain comprising or consisting of SEQ ID NO: 23 or a functional variant thereof, and two different light chains comprising or consisting of SEQ ID NO: 16 and SEQ ID NO: 14 or functional variants thereof.

In a particular embodiment, the bispecific antigen-binding fragment comprises:
(i) a Fab fragment comprising a VH-CH1 heavy chain associated with a VL-CL light chain of patritumab or a functional derivative thereof, and
(ii) a Fab fragment comprising a VH-CH1 heavy chain associated with a VL-CL light chain of cetuximab or a functional or humanized derivative thereof, wherein the N-terminal end of the VH domain of the Fab fragment of patritumab is linked to the C-terminal end of the CH1 domain of the Fab fragment of cetuximab through a polypeptide linker,
wherein the VH-CH1 heavy chain of cetuximab preferably consists of SEQ ID NO: 15, the VL-CL light chain of cetuximab preferably consists of SEQ ID NO: 16, the VH-CH1 heavy chain of patritumab preferably consists of SEQ ID NO: 13, and/or wherein the VL-CL light chain of patritumab preferably consists of SEQ ID NO: 14.

In a preferred embodiment, the bispecific antigen binding fragment comprises one heavy chain comprising or consisting of SEQ ID NO: 24 or a functional variant thereof, and two different light chains comprising or consisting of SEQ ID NO: 16 and SEQ ID NO: 14 or functional variants thereof.

In a particular embodiment, the bispecific antigen-binding fragment comprises:
(i) a Fab fragment comprising a VH-CH1 heavy chain associated with a VL-CL light chain of matuzumab or a functional derivative thereof, and
(ii) a Fab fragment comprising a VH-CH1 heavy chain associated with a VL-CL light chain of patritumab or a functional derivative thereof,
wherein the N-terminal end of the VH domain of the Fab fragment of matuzumab is linked to the C-terminal end of the CH1 domain of the Fab fragment of patritumab through a polypeptide linker,
wherein the VH-CH1 heavy chain of matuzumab preferably consists of SEQ ID NO: 19, the VL-CL light chain of matuzumab preferably consists of SEQ ID NO: 20, the VH-CH1 heavy chain of patritumab preferably consists of SEQ ID NO: 13, and/or wherein the VL-CL light chain of patritumab preferably consists of SEQ ID NO: 14.

In a preferred embodiment, the bispecific antigen binding fragment comprises one heavy chain comprising or consisting of SEQ ID NO: 25 or a functional variant thereof, and two different light chains comprising or consisting of SEQ ID NO: 20 and SEQ ID NO: 14 or functional variants thereof.

In a particular embodiment, the bispecific antigen-binding fragment comprises:
(i) a Fab fragment comprising a VH-CH1 heavy chain associated with a VL-CL light chain of patritumab or a functional derivative thereof, and
(ii) a Fab fragment comprising a VH-CH1 heavy chain associated with a VL-CL light chain of matuzumab or a functional derivative thereof,
wherein the N-terminal end of the VH domain of the Fab fragment of patritumab is linked to the C-terminal end of the CH1 domain of the Fab fragment of matuzumab through a polypeptide linker,
wherein the VH-CH1 heavy chain of matuzumab preferably consists of SEQ ID NO: 19, the VL-CL light chain of matuzumab preferably consists of SEQ ID NO: 20, the VH-CH1 heavy chain of patritumab preferably consists of SEQ ID NO: 13, and/or wherein the VL-CL light chain of patritumab preferably consists of SEQ ID NO: 14.

In a preferred embodiment, the bispecific antigen binding fragment comprises one heavy chain comprising or consisting of SEQ ID NO: 26 or a functional variant thereof, and two different light chains comprising or consisting of SEQ ID NO: 20 and SEQ ID NO: 14 or functional variants thereof.

### Design of the Linkers

A polypeptide linker is used to link the N-terminal end of the VH domain of the Fab fragment of Ab1 to the C-terminal end of the CH1 domain of the Fab fragment of Ab2.

In the context of the present invention, the term “polypeptide linker sequence” is a polypeptide of about 20 to 80 amino acids, preferably between 30 and 60 amino acids, still preferably between 30 and 40 amino acids. The polypeptide linker sequence typically consists of less than 80 amino acids, preferably less than 60 amino acids, still preferably less than 40 amino acids. Advantageously, the linker sequence is “hinge-derived”, which means that the polypeptide linker comprises all or part of the sequence of the hinge region of one or more immunoglobulin(s) selected among IgA, IgG, and IgD, preferably of human origin. It is also designated “hinge-derived polypeptide linker sequence” or “pseudo hinge linker”. Said polypeptide linker may comprise all or part of the sequence of the hinge region of only one immunoglobulin. In this case, said immunoglobulin may belong to the same isotype and subclass as the immunoglobulin from which the adjacent CH1 domain is derived, or to a different isoty45pe or subclass. Alternatively, said polypeptide linker may comprise all or part of the sequences of hinge regions of at least two immunoglobulins of different isotypes or subclasses. In this case, the N-terminal portion of the polypeptide linker, which directly follows the CH1 domain, preferably consists of all or part of the hinge region of an immunoglobulin belonging to the same isotype and subclass as the immunoglobulin from which said CH1 domain is derived.

Optionally, said polypeptide linker may further comprise a sequence of from 2 to 15, preferably of from 5 to 10 N-terminal amino acids of the CH2 domain of an immunoglobulin.

In some cases, sequences from native hinge regions can be used; in other cases point mutations can be brought to these sequences, in particular the replacement of one or more cysteine residues in native IgG1, IgG2 or IgG3 hinge sequences by alanine or serine, in order to avoid unwanted intra-chain or inter-chains disulfide bonds.

In a particular embodiment, the polypeptide linker sequence comprises or consists of amino acid sequence EPKX1CDKX2HX3X4PPX5PAPELLGGPX6X7PPX8PX9PX10GG (SEQ ID NO: 33), wherein X1, X2, X3, X4, X5, X6, X7, X8, X9, X10, identical or different, are any amino acid.

In a particular embodiment, X1, X2 and X3, identical or different, are Threonine (T) or Serine(S).

In another particular embodiment, X1, X2 and X3, identical or different, are selected from the group consisting of Ala (A), Gly (G), Val (V), Asn (N), Asp (D) and Ile (I), still preferably X1, X2 and X3, identical or different, may be Ala (A) or Gly (G).

Alternatively, X1, X2 and X3, identical or different, may be Leu (L), Glu (E), Gln (Q), Met (M), Lys (K), Arg (R), Phe (F), Tyr (T), His (H), Trp (W), preferably Leu (L), Glu (E), or Gln (Q).

In a particular embodiment, X4 and X5, identical or different, are any amino acid selected from the group consisting of Serine(S), Cysteine (C), Alanine (A), and Glycine (G).

In a preferred embodiment, X4 is Serine(S) or Cysteine (C).

In a preferred aspect, X5 is Alanine (A) or Cysteine (C).

In a particular embodiment, X6, X7, X8, X9, X10, identical or different, are any amino acid other than Threonine (T) or Serine(S). Preferably X6, X7, X8, X9, X10, identical or different, are selected from the group consisting of Ala (A), Gly (G), Val (V), Asn (N), Asp (D) and Ile (I).

Alternatively, X6, X7, X8, X9, X10, identical or different, may be Leu (L), Glu (E), Gln (Q), Met (M), Lys (K), Arg (R), Phe (F), Tyr (T), His (H), Trp (W), preferably Leu (L), Glu (E), or Gln (Q).

In a preferred embodiment, X6, X7, X8, X9, X10, identical or different, are selected from the group consisting of Ala (A) and Gly (G).

In still a preferred embodiment, X6 and X7 are identical and are preferably selected from the group consisting of Ala (A) and Gly (G).

In a preferred embodiment, the polypeptide linker sequence comprises or consists of sequence SEQ ID NO: 33, wherein
X1, X2 and X3, identical or different, are Threonine (T), Serine(S);
X4 is Serine(S) or Cysteine (C);
X5 is Alanine (A) or Cysteine (C);
X6, X7, X8, X9, X10, identical or different, are selected from the group consisting of Ala (A) and Gly (G).

In another preferred embodiment, the polypeptide linker sequence comprises or consists of sequence SEQ ID NO: 33, wherein
X1, X2 and X3, identical or different, are Ala (A) or Gly (G);
X4 is Serine(S) or Cysteine (C);
X5 is Alanine (A) or Cysteine (C);
X6, X7, X8, X9, X10, identical or different, are selected from the group consisting of Ala (A) and Gly (G).

In particular, the polypeptide linker sequence may comprise or consist of a sequence selected from the group consisting of:

(SEQ ID NO: 34)
EPKSCDKTHTSPPAPAPELLGGPAAPPAPAPAGG;
(SEQ ID NO: 35)
EPKSCDKTHTAPPAPAPELLGGPAAPPAPAPAGG;
(SEQ ID NO: 36)
EPKSCDKTHTSPPAPAPELLGGPGGPPGPGPGGG;
(SEQ ID NO: 37)
EPKSCDKTHTSPPAPAPELLGGPAAPPGPAPGGG;
(SEQ ID NO: 38)
EPKSCDKTHTCPPCPAPELLGGPSTPPTPSPSGG;
and
(SEQ ID NO: 39)
EPKSCDKTHTSPPSPAPELLGGPSTPPTPSPSGG.

Preferably, the polypeptide linker sequence comprises or consists of the amino acid sequence of SEQ ID NO: 34 or SEQ ID NO: 35.

In embodiments wherein the antibodies comprise different Fab fragments, the polypeptide linkers separating the Fab fragments can be identical or different.

### 2. Design of Bispecific Antibodies

The invention also relates to a bispecific molecule that comprises two identical antigen-binding arms, each consisting of an antigen-binding fragment as defined above. In a preferred embodiment, the bispecific molecule is a full-length antibody.

If one wishes to obtain an antibody without Fc-mediated effects or an antibody monovalent for each of the two antigens it targets, the antibody will comprise no Fc region. In this case, the two antigen-binding arms can be linked together for instance:
by homodimerization of the antigen-binding arms through the inter-chain disulfide bonds provided by the polypeptide linker(s) separating the Fab fragments; and/or
through the addition at the C-terminal end of each antigen-binding arm, of a polypeptide extension containing cysteine residues allowing the formation of inter-chain disulfide bonds, and homodimerization of said polypeptide extension resulting in a hinge-like structure; by way of non-limiting examples, said polypeptide extension may be for instance a hinge sequence of an IgG1, IgG2 or IgG3;
through a linker, preferably a semi-rigid linker, joining the C-terminal ends of the heavy chains of the two antigen-binding arms to form a single polypeptide chain and maintaining said antigen-binding arms at a sufficient distance between each other.

Alternatively, if effector functions such as antibody-dependent cell-mediated cytotoxicity (ADCC), complement-dependent cytotoxicity (CDC) and/or antibody-dependent phagocytosis (ADP) or bivalent binding for each of the two antigens are desired, a multispecific antibody of the invention can further comprise a Fc domain providing these effector functions. The choice of the Fc domain will depend on the type of desired effector functions.

The invention also provides bispecific tetravalent antibodies, comprising two binding sites to HER-3 and two binding sites to another antigen being either HER-2 or EGFR. More particularly, the invention relates to a bispecific antibody that comprises two identical antigen-binding arms (thereby providing a symmetrical antibody format), wherein each antigen-binding arm comprises one binding site to HER-3 and one binding site to HER-2. The invention also relates to a bispecific antibody that comprises two identical antigen-binding arms (thereby providing a symmetrical antibody format), wherein each antigen-binding arm comprises one binding site to HER-3 and one binding site to EGFR.

In a particular embodiment, each antigen-binding arm consists of a bispecific antigen-binding fragment as defined above.

In a preferred embodiment, a linker connects two pairs of Fab domains in a tetra-Fab bispecific antibody format, the amino acid sequence of which comprises the heavy chain sequences of at least two Fab joined by a linker, followed by a hinge sequence, followed by the Fc sequence, coexpressed with the appropriate light chain sequences.

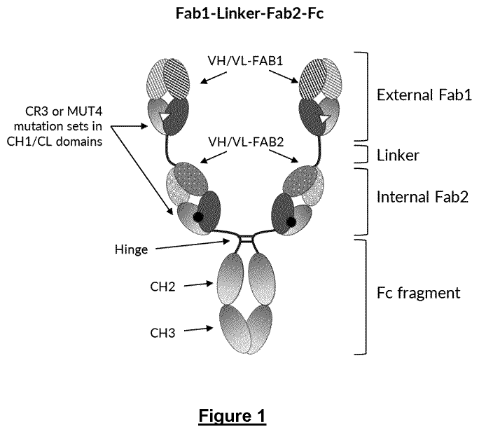

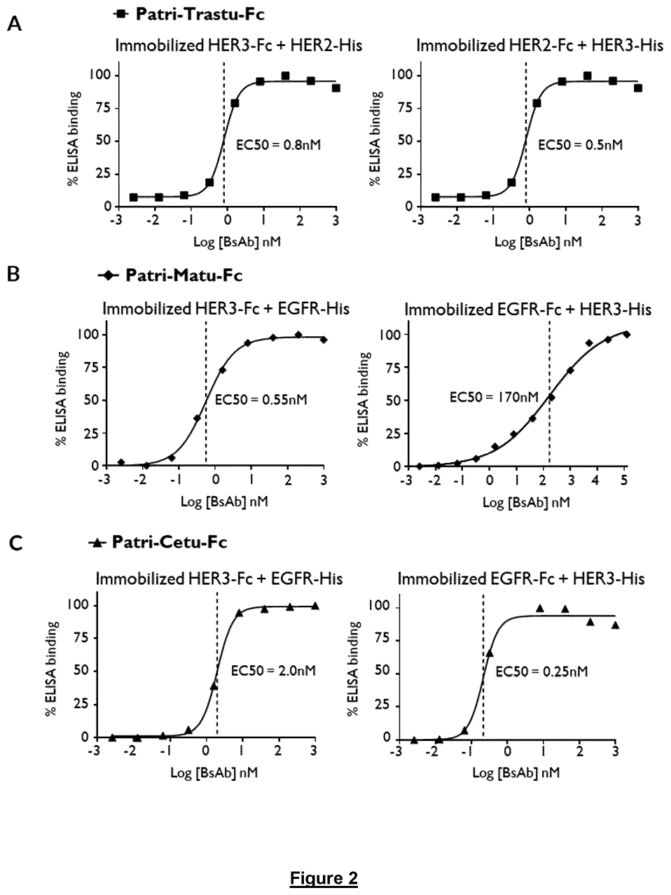

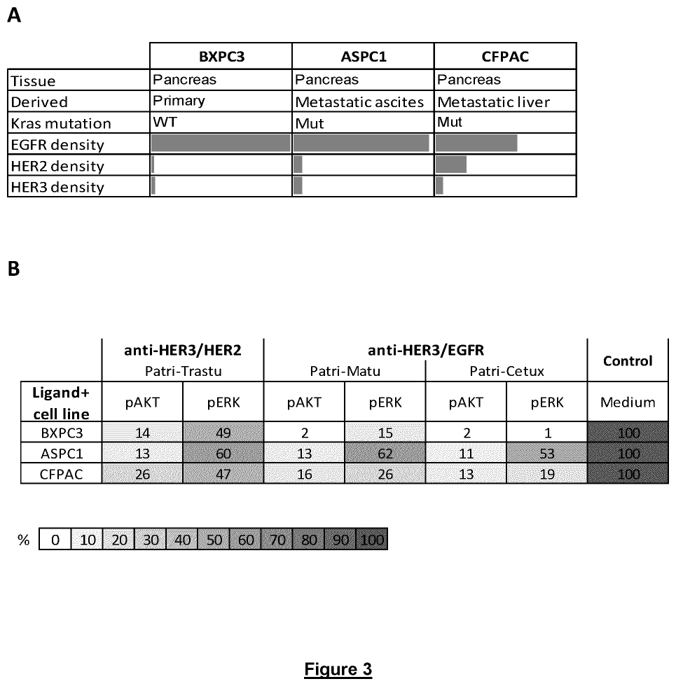

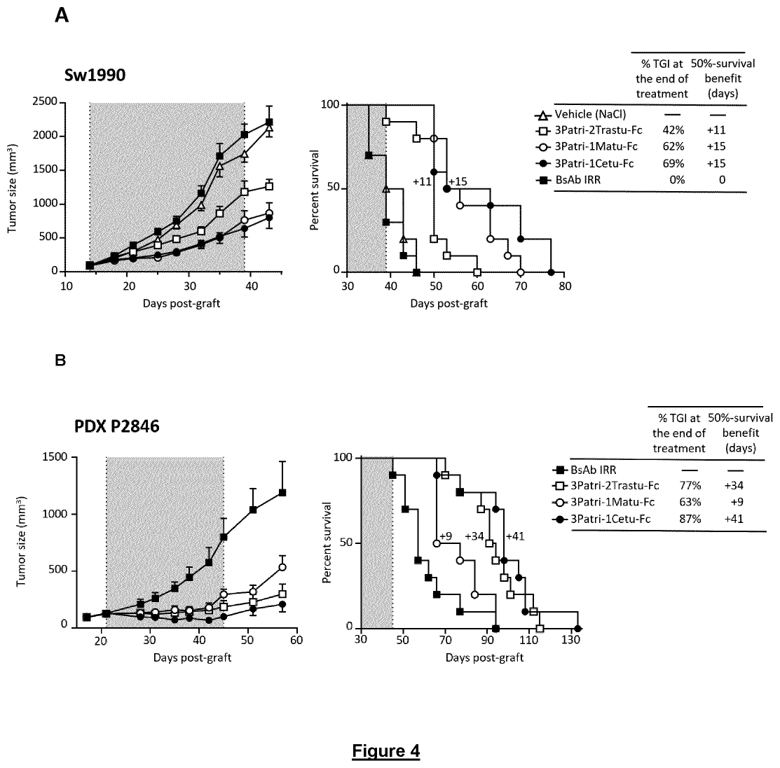

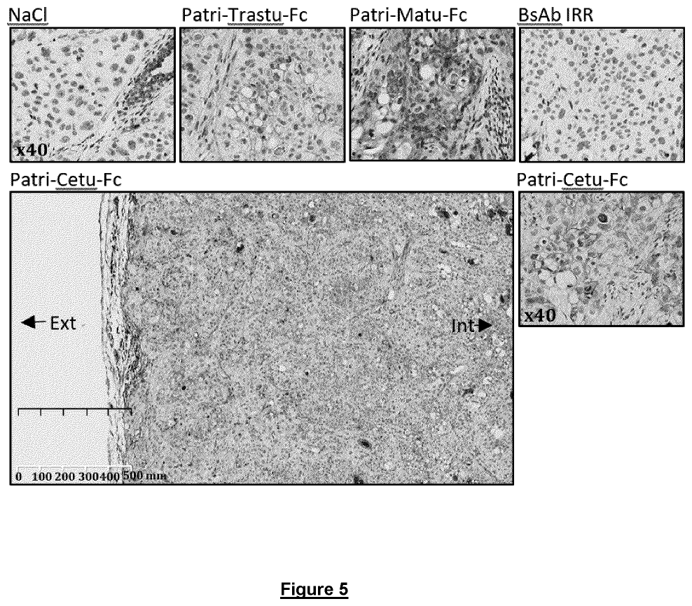

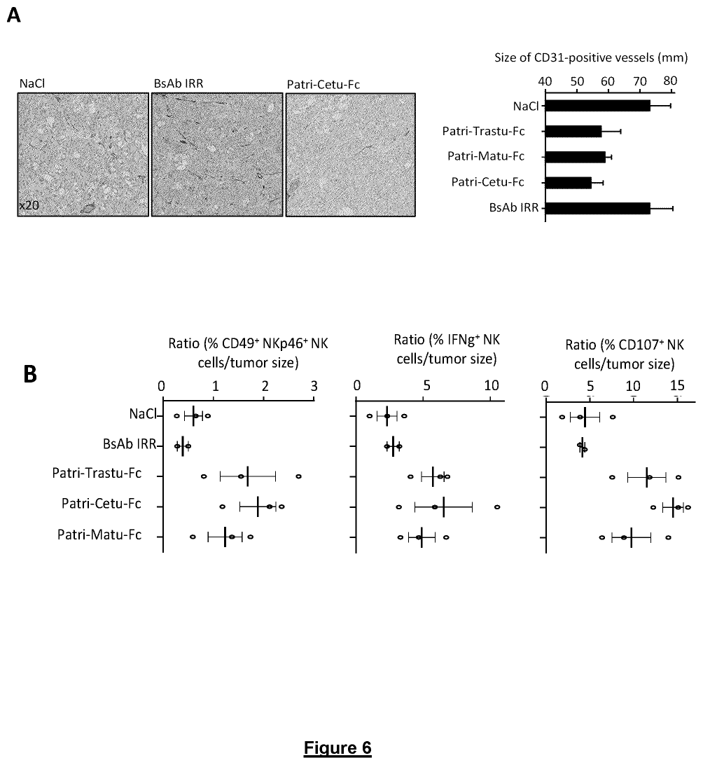

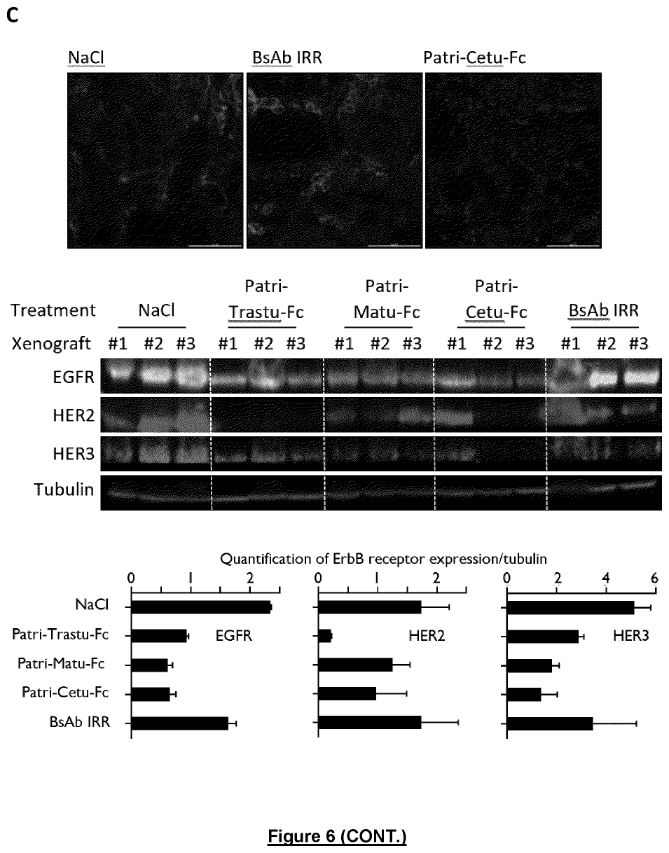

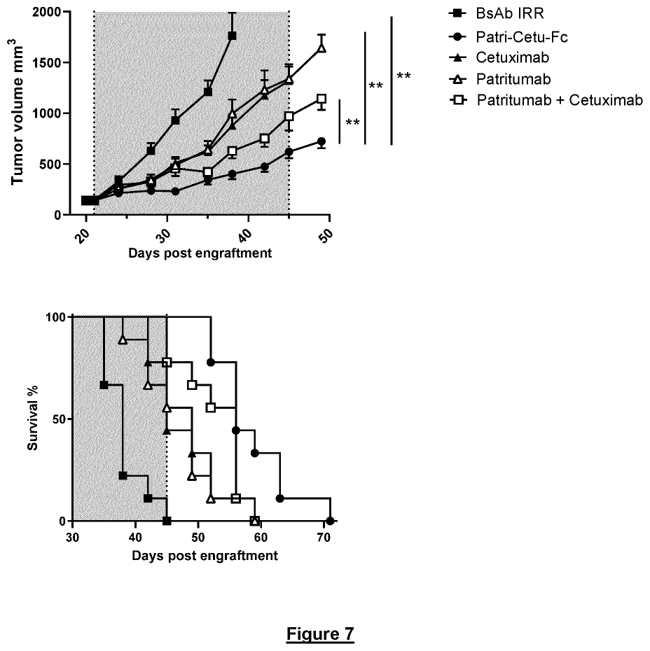

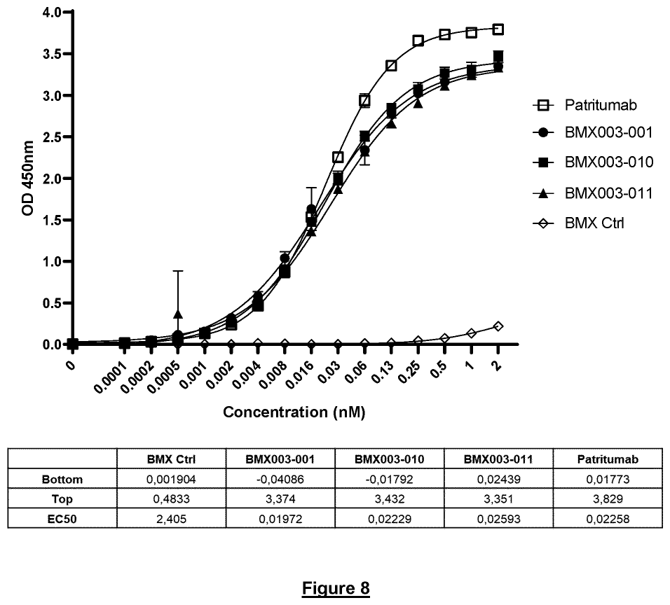

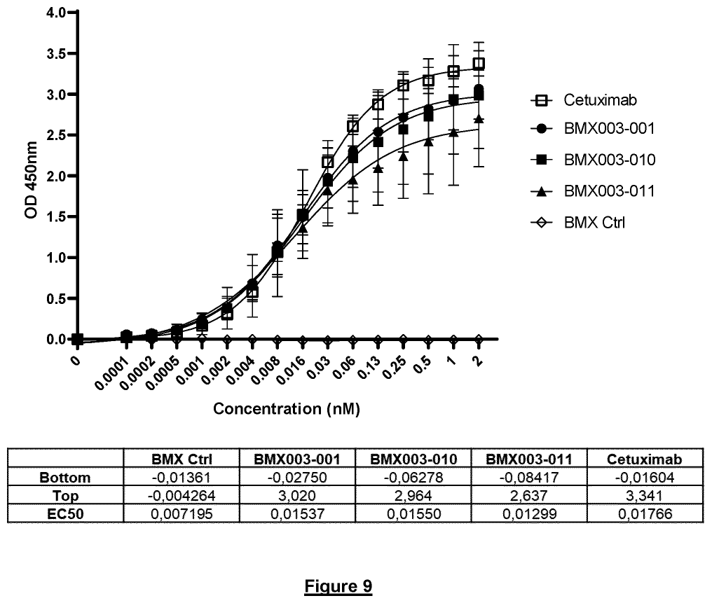

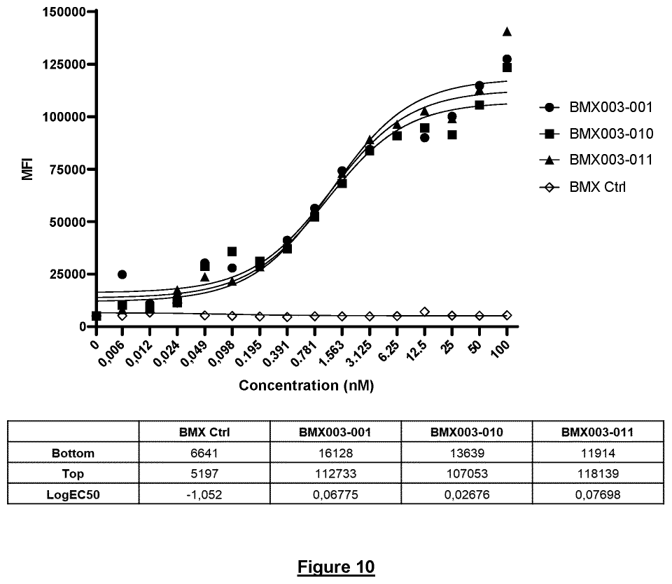

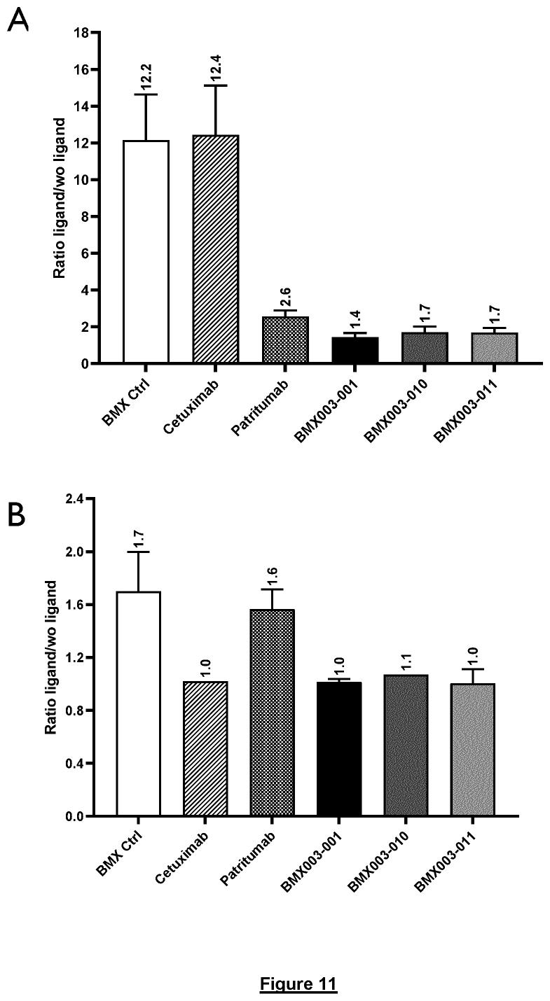

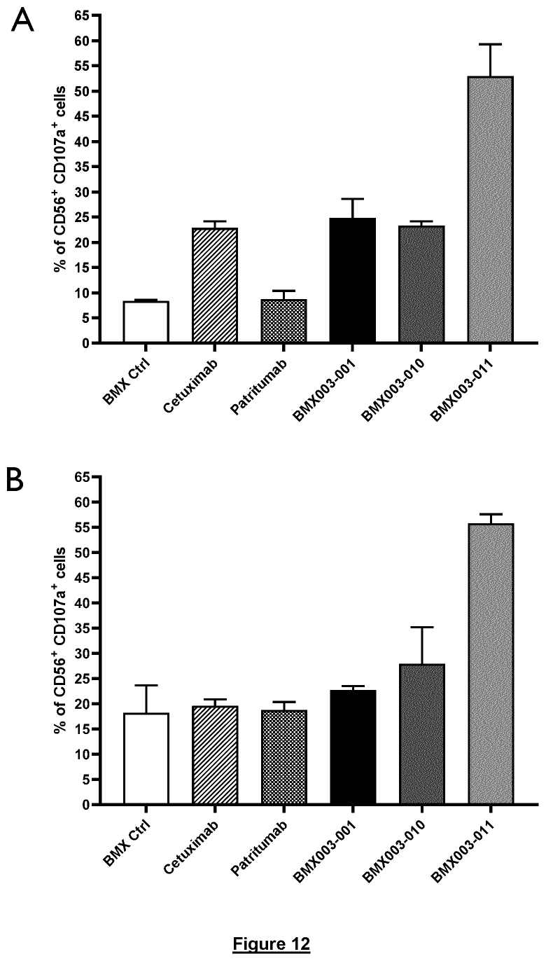

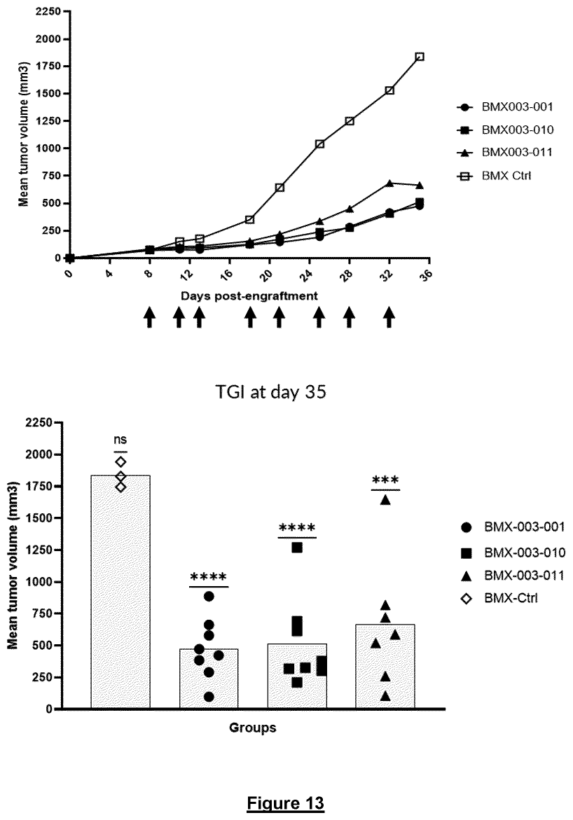

An example of the antibodies of the invention, which have an IgG-like structure, is illustrated in FIG. 1.

In a particular embodiment, the bispecific antibody has an immunoglobulin-like structure, and comprises:
two identical antigen-binding arms, each consisting of a bispecific antigen-binding fragment as described herein, and
a Fc domain, which is preferably functional, i.e. it allows the activation of effector functions such as antibody-dependent cell-mediated cytotoxicity (ADCC), phagocytosis, and complement-dependent cytotoxicity (CDC).

In a particular embodiment, the bispecific antibodies of the invention comprises:
a continuous heavy chain constructed of an Fc (Hinge-CH2-CH3),
followed by Fab heavy chain (CH1-VH) of antibody 1 (Ab1) and the successive Fab heavy chain (CH1-VH) of Antibody 2 (Ab2), the latter joined by the polypeptide linker sequence as described above,
and during protein expression the resulting heavy chain assembles into homo-dimers while the co-expressed two light chains (VL-CL) of Ab1 and two light chains (VL-CL) of Ab2 associate with their cognate heavy chains in order to form the final tandem F (ab)′2-Fc molecule,
   wherein one of Ab1 or Ab2 is patritumab or a functional derivative thereof;
   and the other of Ab1 or Ab2 is selected from the group consisting of trastuzumab or a functional derivative thereof, matuzumab or a functional derivative thereof, and cetuximab or a functional derivative thereof.

It is understood that the hinge domain links the N-terminal end of the CH2 domain to the C-terminal end of the CH1 domain of Ab1, and that the polypeptide linker sequence links the N-terminal end of the VH domain of Ab1 to the C-terminal end of the CH1 domain of Ab2.

In a particular embodiment, the bispecific antibody has an immunoglobulin-like structure, comprising:
two identical antigen-binding arms each consisting of a bispecific antigen-binding fragment as described herein;
the dimerized CH2 and CH3 domains of an immunoglobulin;
the hinge region of an of an immunoglobulin linking the C-terminal ends of CH1 domains of the antigen-binding arms to the N-terminal ends of the CH2 domains.

In a particular embodiment, the CH2 and/or CH3 domain are CH2 and CH3 domains of IgG1 or IgG4 isotype, or mutated derivative thereof. Preferably, the CH2 domain is the CH2 domain of human IgG1 (SEQ ID NO: 41). Preferably, the CH3 domain is the CH3 domain of human IgG1 of G1m (3) allotype (SEQ ID NO: 42). In another particular embodiment, the CH3 domain is the CH3 domain of human IgG1 of G1m (1,17) allotype.

In a particular embodiment, the hinge region is the hinge region of an IgA, IgG1, IgG4, IgD, or a mutated derivative thereof, linking the C-terminal ends of CH1 domains of the antigen-binding arms to the N-terminal ends of the CH2 domains. In a particular embodiment, the hinge regions is the hinge region of IgG4, with S228P substitution. Preferably, the hinge region is the region of IgG1 of SEQ ID NO: 40.

In a particular embodiment, the bispecific antibody has an immunoglobulin-like structure, and comprises a Fc domain, which may be a wild-type immunoglobulin Fc domain, or a mutated derivative thereof. In a particular embodiment, the Fc domain is a mutated derivative of IgG1 Fc domain, or a mutated derivative of IgG4 Fc domain. In a particular embodiment, said mutated derivative is a Fc domain having a modified affinity for at least one Fc gamma receptor (FcgR) in comparison with a parent Fc domain.

In a particular embodiment, the bispecific antibody has an immunoglobulin-like structure, and comprises a Fc domain, such as IgG1 or IgG4 Fc domain, having a reduced binding to Fc gamma receptors when compared to wild-type Fc domain, leading to a bispecific antibody having a reduced effector function.

In a particular embodiment, the bispecific antibody has an immunoglobulin-like structure, and comprises a Fc domain, such as IgG1 or IgG4 Fc domain, that shows no binding, or substantially no binding, to Fc gamma receptors when compared to the wild-type Fc domain, leading to a bispecific antibody having a silenced or reduced effector function.

Said Fc domain may comprise one or more mutation(s) reducing or eliminating its binding to Fc gamma receptors, such as mutation(s) in CH2 and/or CH3 domains. Said mutation(s) include amino acid substitution, insertion and/or deletion. Illustrative mutations reducing or eliminating the binding activity of the Fc domain to Fc gamma receptors include, but are not limited to: L/F234A, L235A/E, G236R/del, G237A, P238S, D265A, H/Q268A, L328R, P329G, S/A330R/S, and/or P331S. All mutated residues in the Fc domain are herein numbered according to the EU nomenclature convention.

For clarification purpose, “L/F234A” denotes the substitution of Leucine or Phenylalanine (whichever is present in IgG1 or IgG4, respectively, at position 234) by Alanine. For clarification purpose, “L235A/E” denotes the replacement of Leucine at position 235 by either Alanine or Glutamic acid. For clarification purpose, “G236R/del” denotes the replacement of Glycine at position 236 by either Arginine, or the deletion of the Glycine residue at position 236. For clarification purpose, “A/S330R/S” denotes the replacement of Alanine or Serine (whichever is present in IgG1 or IgG4, respectively, at position 330) by either Arginine or Serine.

Illustrative mutations reducing or eliminating the binding activity of the IgG1 Fc domain to Fc gamma receptors include, but are not limited to: L234A, L235A/E, G236R/del, G237A, P238S, D265A, H268A, L328R, P329G, A330R/S, and/or P331S. Illustrative mutations reducing or eliminating the binding activity of the IgG4 Fc domain to Fc gamma receptors include, but are not limited to: F234A, L235A/E, G236R/del, G237A, P238S, D265A, Q268A, L328R, P329G, and/or S330R.

In another particular embodiment, the bispecific antibody has an immunoglobulin-like structure, and comprises a Fc domain, such as IgG1 or IgG4 Fc domain, having an increased binding to Fc gamma receptors when compared to wild-type Fc domain, leading to a bispecific antibody having an improved effector function.

Said Fc domain may comprise one or more mutation(s) increasing its binding to Fc gamma receptors, such as mutation(s) in CH2 and/or CH3 domains. Illustrative mutations increasing the binding activity of the Fc domain, such as IgG1 or IgG4 Fc domain, to Fc gamma receptors include, but are not limited to: S239D, 1332E, S298A, E333A, K334A, D280H, K290S, S298D, F243L, R292P, Y300L, V3051, P396L, A330L, G236A, L234Y, G236W, and/or S298A. In a preferred embodiment, the Fc domain, such as IgG1 or IgG4 Fc domain comprises mutations S239D and 1332E.

In a particular embodiment, the Fc domain, such as IgG1 or IgG4 Fc domain, has a reduced fucose content in the Fc glycan at position 297 in the CH2 domain.

According to a particular embodiment, the Fc domain, such as IgG1 or IgG4 Fc domain, has N-glycans on the glycosylation site (Asn 297), said N-glycans having a degree of fucosylation lower than 65%, preferably lower than 60%, preferably lower than 55%, preferably lower than 50%, more preferably lower than 45%, preferably lower than 40%, preferably lower than 35%, preferably lower than 30%, preferably lower than 25%, preferably lower than 20%, preferably lower than 10%.

According to a more particular embodiment, the Fc domain has N-glycans on the glycosylation site (Asn 297), said N-glycans having a degree of fucosylation equal to 0%. The invention thus provides a bispecific antibody comprising a Fc domain having N-glycans on the glycosylation site Asn297 thereof, characterized in that said N-glycans of the Fc domain are fucose-free.

Advantageously, the Fc domain having a modified glycosylation at the glycosylation site at position 297, in particular a low fucosylation, shows an increased binding to Fc-gamma receptors.

In a particular embodiment, the Fc domain comprises, preferably consists of:
the hinge domain of SEQ ID NO: 40; and
the CH2-CH3 domain of SEQ ID NO: 43.

In a particular embodiment, the Fc domain comprises, preferably consists of SEQ ID NO: 44, or a functional variant having at least 80%, 90%, 95%, 96%, 97%, 98% or at least 99% sequence identity with SEQ ID NO: 44. In another particular embodiment, the Fc domain comprises, preferably consists of SEQ ID NO: 82, or a functional variant having at least 80%, 90%, 95%, 96%, 97%, 98% or at least 99% sequence identity with SEQ ID NO: 82.

A particular embodiment relates to a bispecific antibody comprising two heavy chains and four light chains, wherein each heavy chain comprises:
a. a Fc region of an immunoglobulin comprising Hinge-CH2-CH3 domains or mutated derivatives as described above,
b. which Fc region is linked to Fab CH1-VH heavy chain of antibody 1 (Ab1) by said hinge domain,
C. which in turn is linked to Fab CH1-VH heavy chain of antibody 2 (Ab2), by a polypeptide linker sequence, wherein the polypeptide linker sequence links the N-terminus of said VH domain of Ab1 with the C-terminus of said CH1 domain of Ab2,
wherein the four light chains comprise two Fab CL-VL light chains of Ab1 and two Fab CL-VL light chains of Ab2 associated with their cognate heavy chain domains;
wherein one of Ab1 or Ab2 is patritumab or a functional derivative thereof as described above; and the other of Ab1 or Ab2 is selected from the group consisting of trastuzumab or a functional derivative thereof, matuzumab or a functional derivative thereof, and cetuximab or a functional or humanized derivative thereof as described above.

A particular embodiment relates to a bispecific antibody comprising two heavy chains and four light chains, wherein each heavy chain comprises:
a. a Fc region of an immunoglobulin comprising Hinge-CH2-CH3 domains or mutated derivatives as described above,
b. which Fc region is linked to Fab CH1-VH heavy chain of antibody 1 (Ab1) by said hinge domain,
c. which in turn is linked to Fab CH1-VH heavy chain of antibody 2 (Ab2), by a polypeptide linker sequence, wherein the polypeptide linker sequence links the N-terminus of said VH domain of Ab1 with the C-terminus of said CH1 domain of Ab2,
wherein the four light chains comprise two Fab CL-VL light chains of Ab1 and two Fab CL-VL light chains of Ab2 associated with their cognate heavy chain domains;
wherein Ab2 is patritumab or a functional derivative thereof as described above;
and Ab1 is selected from the group consisting of trastuzumab or a functional derivative thereof, matuzumab or a functional derivative thereof, and cetuximab or a functional or humanized derivative thereof as described above.

In a particular embodiment, the bispecific antibody comprises, preferably consists of:
a) two heavy chains, each comprising, preferably consisting of SEQ ID NO: 27 or a mutative derivative thereof and
b) four light chains, two comprising, preferably consisting of SEQ ID NO: 14 or a mutative derivative thereof, the two others comprising, preferably consisting of SEQ ID NO: 18 or a mutative derivative thereof.

The heavy chain of SEQ ID NO: 27 comprises:
VH of patritumab (SEQ ID NO: 5),
CH1 domain (human IgG1 of G1m (1,17) allotype with the mutation T192E) of patritumab Fab (SEQ ID NO: 1),
AP linker (SEQ ID NO: 34),
VH of trastuzumab (SEQ ID NO: 9),
CH1 domain (human IgG1 of G1m (1,17) allotype with mutations L143Q and S188V) of trastuzumab Fab (SEQ ID NO: 2),
Hinge of human IgG1 (SEQ ID NO: 40),
CH2 domain of human IgG1 (SEQ ID NO: 41),
CH3 domain of human IgG1 of G1m (3) allotype (SEQ ID NO: 42).

In a particular embodiment, the bispecific antibody comprises, preferably consists of:
a) two heavy chains, each comprising, preferably consisting of SEQ ID NO: 28 or a mutative derivative thereof and
b) four light chains, two comprising, preferably consisting of SEQ ID NO: 14 or a mutative derivative thereof, the two others comprising, preferably consisting of SEQ ID NO: 18 or a mutative derivative thereof.

The heavy chain of SEQ ID NO: 28 comprises:
VH of trastuzumab (SEQ ID NO: 9),
CH1 domain (human IgG1 of G1m (1,17) allotype with mutations L143Q and S188V) of trastuzumab Fab (SEQ ID NO: 2),
AP linker (SEQ ID NO: 34),
VH of patritumab (SEQ ID NO: 5),
CH1 domain (human IgG1 of G1m (1,17) allotype with the mutation T192E) of patritumab Fab (SEQ ID NO: 1),
Hinge of human IgG1 (SEQ ID NO: 40),
CH2 domain of human IgG1 (SEQ ID NO: 41),
CH3 domain of human IgG1 of G1m (3) allotype (SEQ ID NO: 42).

In a particular embodiment, the bispecific antibody comprises, preferably consists of:
a) two heavy chains, each comprising, preferably consisting of SEQ ID NO: 29 or a mutative derivative thereof and
b) four light chains, two comprising, preferably consisting of SEQ ID NO: 14 or a mutative derivative thereof, the two others comprising, preferably consisting of SEQ ID NO: 16 or a mutative derivative thereof.

The heavy chain of SEQ ID NO: 29 comprises:
VH of patritumab (SEQ ID NO: 5),
CH1 domain (human IgG1 of G1m (1,17) allotype with the mutation T192E) of patritumab Fab (SEQ ID NO: 1),
AP linker (SEQ ID NO: 34),
VH of cetuximab (SEQ ID NO: 7),
CH1 domain (human IgG1 of G1m (1,17) allotype with mutations L143Q and S188V) of trastuzumab Fab (SEQ ID NO: 2),
Hinge of human IgG1 (SEQ ID NO: 40),
CH2 domain of human IgG1 (SEQ ID NO: 41),
CH3 domain of human IgG1 of G1m (3) allotype (SEQ ID NO: 42).

In a particular embodiment, the bispecific antibody comprises, preferably consists of:
a) two heavy chains, each comprising, preferably consisting of SEQ ID NO: 30 or a mutative derivative thereof, and
b) four light chains, two comprising, preferably consisting of SEQ ID NO: 14 or a mutative derivative thereof, the two others comprising, preferably consisting of SEQ ID NO: 16 or a mutative derivative thereof.

The heavy chain of SEQ ID NO: 30 comprises:
VH of cetuximab (SEQ ID NO: 7),
CH1 domain (human IgG1 of G1m (1,17) allotype with mutations L143Q and S188V) of cetuximab Fab (SEQ ID NO: 2),
AP linker (SEQ ID NO: 34),
VH of patritumab (SEQ ID NO: 5),
CH1 domain (human IgG1 of G1m (1,17) allotype with the mutation T192E) of patritumab Fab (SEQ ID NO: 1),
Hinge of human IgG1 (SEQ ID NO: 40),
CH2 domain of human IgG1 (SEQ ID NO: 41),
CH3 domain of human IgG1 of G1m (3) allotype (SEQ ID NO: 42).

In a particular embodiment, the bispecific antibody comprises, preferably consists of:
a) two heavy chains, each comprising, preferably consisting of SEQ ID NO: 31 or a mutative derivative thereof, and
b) four light chains, two comprising, preferably consisting of SEQ ID NO: 14 or a mutative derivative thereof, the two others comprising, preferably consisting of SEQ ID NO: 20 or a mutative derivative thereof.

The heavy chain of SEQ ID NO:31 comprises:
VH of patritumab (SEQ ID NO: 5),
CH1 domain (human IgG1 of G1m (1,17) allotype with the mutation T192E) of patritumab Fab (SEQ ID NO: 1),
AP linker (SEQ ID NO: 34),
VH of matuzumab (SEQ ID NO: 11),
CH1 domain (human IgG1 of G1m (1,17) allotype with mutations L143Q and S188V) of trastuzumab Fab (SEQ ID NO: 2),
Hinge of human IgG1 (SEQ ID NO: 40),
CH2 domain of human IgG1 (SEQ ID NO: 41),
CH3 domain of human IgG1 of G1m (3) allotype (SEQ ID NO: 42).

In a particular embodiment, the bispecific antibody comprises, preferably consists of:
a) two heavy chains, each comprising, preferably consisting of SEQ ID NO: 32 or a mutative derivative thereof, and
b) four light chains, two comprising, preferably consisting of SEQ ID NO: 14 or a mutative derivative thereof, the two others comprising, preferably consisting of SEQ ID NO: 20 or a mutative derivative thereof.

The heavy chain of SEQ ID NO: 32 comprises:
VH of matuzumab (SEQ ID NO: 11),
CH1 domain (human IgG1 of G1m (1,17) allotype with mutations L143Q and S188V) of cetuximab Fab (SEQ ID NO: 2),
AP linker (SEQ ID NO: 34),
VH of patritumab (SEQ ID NO: 5),
CH1 domain (human IgG1 of G1m (1,17) allotype with the mutation T192E) of patritumab Fab (SEQ ID NO: 1),
Hinge of human IgG1 (SEQ ID NO: 40),
CH2 domain of human IgG1 (SEQ ID NO: 41),
CH3 domain of human IgG1 of G1m (3) allotype (SEQ ID NO: 42).

The light chain of SEQ ID NO: 14 comprises:
VL of patritumab (SEQ ID NO: 6),
CKappa domain with mutations S114A and N137K (SEQ ID NO: 3).

The light chain of SEQ ID NO: 16 comprises:
VL of cetuximab (SEQ ID NO: 8),
CKappa domain with mutations V133T and S176V (SEQ ID NO: 4).

The light chain of SEQ ID NO: 18 comprises:
VL of trastuzumab (SEQ ID NO: 10),
CKappa domain with mutations V133T and S176V (SEQ ID NO: 4).

The light chain of SEQ ID NO: 20 comprises:
VL of matuzumab (SEQ ID NO: 12),
CKappa domain with mutations V133T and S176V (SEQ ID NO: 4).

In a particular embodiment, the bispecific antibody comprises, preferably consists of:
a) two heavy chains, each comprising, preferably consisting of SEQ ID NO: 85 or a mutative derivative thereof, and
b) four light chains, two comprising, preferably consisting of SEQ ID NO: 14 or a mutative derivative thereof, the two others comprising, preferably consisting of SEQ ID NO: 84 or a mutative derivative thereof.

In a particular embodiment, the bispecific antibody comprises, preferably consists of:
a) two heavy chains, each comprising, preferably consisting of SEQ ID NO: 86 or a mutative derivative thereof, and
b) four light chains, two comprising, preferably consisting of SEQ ID NO: 14 or a mutative derivative thereof, the two others comprising, preferably consisting of SEQ ID NO: 84 or a mutative derivative thereof.

### 3. Production of the Bispecific Antibodies

The skilled person may refer to international patent application WO2013/005194, herein incorporated by reference, for general techniques of expressing multispecific antibodies.

Also herein described is a polynucleotide comprising a sequence encoding a protein chain of the molecule or antibody of the invention. Said polynucleotide may also comprise additional sequences: in particular it may advantageously comprise a sequence encoding a leader sequence or signal peptide allowing secretion of said protein chain. Host-cells transformed with said polynucleotide are also disclosed.

Typically, the amino acid sequences of the bispecific antibody are used to design the DNA sequences, optionally after codon optimization for mammalian expression. For the heavy chain, the DNAs encoding signal peptides, variable region and constant CH1 domain of Fab1 followed the hinge linker and variable region and constant CH1 domain of Fab2 with flanking sequences for restriction enzyme digestion are synthesized. For the light chain, the DNAs encoding signal peptides and variable and constant Kappa regions are synthesized.

Nucleic acids encoding heavy and light chains of the bispecific antigen-binding fragments or antibodies of the invention are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Such control sequences include a signal sequence, a promoter, an enhancer, and a transcription termination sequence. Expression vectors are typically replicable in the host organisms either as episomes or as an integral part of the host chromosomal DNA. Commonly, expression vectors will contain selection markers, e.g., tetracycline or neomycin, to permit detection of those cells transformed with the desired DNA sequences.

In one example, both the heavy and light chain-coding sequences (e.g., sequences encoding a VH and a VL, a VH-CH1 and a VL-CL, or a full-length heavy chain and a full-length light chain) are included in one expression vector. n another example, each of the heavy and light chains of the antibody is cloned into an individual vector. In the latter case, the expression vectors encoding the heavy and light chains can be co-transfected into one host cell for expression of both chains, which can be assembled to form intact antibodies either in vivo or in vitro. Alternatively, the expression vector encoding the heavy chain and that or those encoding the light chains can be introduced into different host cells for expression each of the heavy and light chains, which can then be purified and assembled to form intact antibodies in vitro.

In a particular embodiment, a host cell is co-transfected with three independent expression vectors, such as plasmids, leading to the coproduction of all three chains (namely the heavy chain HC, and two light chains LC1 and LC2, respectively) and to the secretion of the bispecific antibody. More especially the three vectors may be advantageously used in a following molecular ratio of 2:1:1 (HC:LC1:LC2).

The recombinant vectors for expression the antibodies described herein typically contain a nucleic acid encoding the antibody amino acid sequences operably linked to a promoter, either constitutive or inducible. The vectors can be suitable for replication and integration in prokaryotes, eukaryotes, or both. Typical vectors contain transcription and translation terminators, initiation sequences, and promoters useful for regulation of the expression of the nucleic acid encoding the antibody. The vectors optionally contain generic expression cassettes containing at least one independent terminator sequence, sequences permitting replication of the cassette in both eukaryotes and prokaryotes, i.e., shuttle vectors, and selection markers for both prokaryotic and eukaryotic systems.

Bispecific antibodies as described herein may be produced in prokaryotic or eukaryotic expression systems, such as bacteria, yeast, filamentous fungi, plant, insect (e.g. using a baculovirus vector), and mammalian cells. It is not necessary that the recombinant antibodies of the invention are glycosylated or expressed in eukaryotic cells; however, expression in mammalian cells is generally preferred. Examples of useful mammalian host cell lines are human embryonic kidney line (293 cells), baby hamster kidney cells (BHK cells), Chinese hamster ovary cells/− or + DHFR (CHO, CHO-S, CHO-DG44, Flp-in CHO cells), African green monkey kidney cells (VERO cells), and human liver cells (Hep G2 cells).

Mammalian tissue cell culture is preferred to express and produce the polypeptides because a number of suitable host cell lines capable of secreting intact immunoglobulins have been developed in the art, and include the CHO cell lines, various Cos cell lines, Hela cells, preferably myeloma cell lines (such as NS0), or transformed B-cells or hybridomas.

In a most preferred embodiment, the bispecific antibodies of the invention are produced by using a CHO cell line, most advantageously CHO-S or CHO-DG-44 cell lines or their derivatives. Expression vectors for these cells can include expression control sequences, such as an origin of replication, a promoter, and an enhancer, and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. The vectors containing the polynucleotide sequences of interest (e.g., the heavy and light chain encoding sequences and expression control sequences) can be transferred into the host cell by well-known methods, which vary depending on the type of cellular host. For example calcium phosphate treatment or electroporation may be used for other cellular hosts. (See generally Sambrook et al., Molecular Cloning: A Laboratory Manual (Cold Spring Harbor Press, 2nd ed., 1989). When heavy and light chains are cloned on separate expression vectors, the vectors are co-transfected to obtain expression and assembly of intact immunoglobulins.

Host cells are transformed or transfected with the vectors (for example, by chemical transfection or electroporation methods) and cultured in conventional nutrient media (or modified as appropriate) for inducing promoters, selecting transformants, or amplifying the genes encoding the desired sequences.

The expression of the antibodies may be transient or stable.

Preferably, the bispecific antibodies are produced by the methods of stable expression, in which cell lines stably transfected with the DNA encoding all polypeptide chains of a bispecific antibody, are capable of sustained expression, which enables manufacturing of therapeutics. For instance stable expression in a CHO cell line is particularly advantageous.

Once expressed, the whole antibodies, their dimers, individual light and heavy chains, or other immunoglobulin forms of the present invention can be further isolated or purified to obtain preparations that substantially homogeneous for further assays and applications. Standard protein purification methods known in the art can be used. For example, suitable purification procedures may include fractionation on immunoaffinity or ion-exchange columns, ethanol precipitation, high-performance liquid chromatography (HPLC), sodium dodecyl sulfate polyacrylamide gel electrophoresis (SDS-PAGE), ammonium sulfate precipitation, and gel filtration (see generally Scopes, Protein Purification (Springer-Verlag, N.Y., 1982). Substantially pure immunoglobulins of at least about 90 to 95% homogeneity are preferred, and 98 to 99% or more homogeneity most preferred, for pharmaceutical uses.

In vitro production allows scale-up to give large amounts of the desired bispecific antibodies of the invention. Such methods may employ homogeneous suspension culture, e.g. in an airlift reactor or in a continuous stirrer reactor, or immobilized or entrapped cell culture, e.g. in hollow fibers, microcapsules, on agarose microbeads or ceramic cartridges.

### 4. Therapeutic Uses

The bispecific antibodies of the invention have been shown to induce tumor growth inhibition.

The bispecific antigen binding fragment or antibody of the invention is useful as a medicament, in particular in treating a cancer.

The term “cancer” as used herein includes any cancer, especially pancreatic cancer and any other cancer characterized by HER3, EGFR or HER2 expression or overexpression, and especially those cancers characterized by co-expression of both HER3 and EGFR or HER3 and HER2.

In some embodiments, the cancer comprises cells with a wild-type KRAS gene.

Examples of cancers are solid tumors such as pancreatic cancer, head and neck cancer, including squamous cell carcinoma, colorectal cancer, breast cancer, lung cancer, gastric cancer, esophageal cancer, ovarian cancer.

Preferably, the cancer is a pancreatic cancer.

It is thus described a method of treatment of a patient suffering from cancer by administering an antibody according to the invention to said patient in the need of such treatment. Another aspect of the invention is thus the use of the bispecific antibodies according to the invention for the manufacture of a medicament for the treatment of cancer.

One aspect of the invention is a pharmaceutical composition comprising a bispecific molecule according to the invention. Another aspect of the invention is the use of a bispecific molecule according to the invention for the manufacture of a pharmaceutical composition. A further aspect of the invention is a method for the manufacture of a pharmaceutical composition comprising a bispecific molecule according to the invention.

In another aspect, the present invention provides a composition, e.g. a pharmaceutical composition, containing a bispecific molecule as defined herein, formulated together with a pharmaceutical carrier.

As used herein, “pharmaceutical carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g. by injection or infusion).

A composition of the present invention can be administered by a variety of methods known in the art. The route and/or mode of administration will vary depending upon the desired results.

To administer the bispecific molecule or antibody of the invention by certain routes of administration, it may be necessary to coat the bispecific molecule or antibody of the invention with, or co-administer the bispecific molecule or antibody of the invention with a material to prevent its inactivation. For example, the bispecific molecule or antibody of the invention may be administered to a subject in an appropriate carrier, for example, liposomes, or a diluent. Pharmaceutically acceptable diluents include saline and aqueous buffer solutions. Pharmaceutical carriers include sterile aqueous solutions or dispersions and sterile powders for the extemporaneous preparation of sterile injectable solutions or dispersion. The use of such media and agents for pharmaceutically active substances is known in the art.

These compositions may also contain adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol, sorbic acid, and the like. It may also be desirable to include isotonic agents, such as sodium chloride into the compositions. In addition, prolonged absorption of the injectable pharmaceutical form may be brought about by the inclusion of agents which delay absorption.

Actual dosage levels of the active ingredients in the pharmaceutical compositions of the present invention may be varied so as to obtain an amount of the active ingredient, which is effective to achieve the desired therapeutic response for a particular patient, composition, and mode of administration, without being toxic to the patient.

The selected dosage level will depend upon a variety of pharmacokinetic factors including the activity of the particular compositions of the present invention employed, the route of administration, the time of administration, the duration of the treatment, other drugs, compounds and/or materials used in combination with the particular compositions employed, the age, sex, weight, condition, general health and prior medical history of the patient being treated, and like factors well known in the medical arts. For example the bispecific molecule or antibody of the invention can be administrated at a dosage of 0.2-20 mg/kg from 3 times/week to 1 time/month.

The present invention, thus generally described above, will be understood more readily by reference to the following examples, which are provided by way of illustration and are not intended to be limiting the instant invention.

### EXAMPLES

### Example 1. Preparation of the Bispecific Antibodies of the Invention

The bispecific antibodies (BsAbs) of the invention comprise 2 heavy chains and four light chains. In particular, the BsAbs have 4 antigen-binding sites (Fabs) with 2 Fabs from Ab1 and 2 other Fabs from Ab2 (FIG. 1). Thus, they have a double binding capacity for each of the 2 target antigens (tetravalence). The distal to the Fc domain Fabs are termed “external Fabs” and the proximal to the Fc domain Fabs are termed “internal Fabs” (FIG. 1).

6 antibodies were designed based on 4 parental antibodies: Patritumab (anti-HER3), atezolizumab (anti-PDL1), cetuximab (anti-EGFR) and matuzumab (anti-EGFR). The nomenclature and the sequences of heavy and light chains of each construct are detailed in Table 1:

TABLE 1
description of the bispecific antibody constructs

| | | | | Light | Light |
|---|---|---|---|---|---|
| | | | | chains | chains |
| | External | Internal | Heavy | (external | (internal |
| Name | Fabs | Fabs | chains | Fabs) | Fabs) |
| Patri-trastu-Fc | Patritumab | Trastuzumab | SEQ ID NO: 27 | SEQ ID NO: 14 | SEQ ID NO: 18 |
| Trastu-Patri-Fc | Trastuzumab | Patritumab | SEQ ID NO: 28 | SEQ ID NO: 18 | SEQ ID NO: 14 |
| Patri-Cetu-Fc | Patritumab | Cetuximab | SEQ ID NO: 29 | SEQ ID NO: 14 | SEQ ID NO: 16 |
| Cetu-Patri-Fc | Cetuximab | Patritumab | SEQ ID NO: 30 | SEQ ID NO: 16 | SEQ ID NO: 14 |
| Patri-Matu-Fc | Patritumab | Matuzumab | SEQ ID NO: 31 | SEQ ID NO: 14 | SEQ ID NO: 20 |
| Matu-Patri-Fc | Matuzumab | Patritumab | SEQ ID NO: 32 | SEQ ID NO: 20 | SEQ ID NO: 14 |

As shown in FIG. 1, each bispecific antibody comprises:
2 identical heavy chains, wherein each heavy chain comprises (from N-ter to C-ter): the VH domain and the CH1 domain of the external Fab, a linker, the VH domain and the CH1 domain of the internal Fab, a constant Fc domain (comprising Hinge-Ch2-Ch3).
2 identical light chains of the external Fabs, wherein each light chain comprises (from N-ter to C-ter): the VL domain and the CL domain of the external Fab.
2 identical light chains of the internal Fabs, wherein each light chain comprises (from N-ter to C-ter): the VL domain and the CL domain of the internal Fab.

Details on the sequences that constitute the heavy and light chains of each construct are provided in Table 2.

TABLE 2
details on the sequences of each bispecific construct.

| | Heavy chain | | | | | | Light chain | | Light chain | |
|---|---|---|---|---|---|---|---|---|---|---|
| | External Fab | | | Internal Fab | | | (external Fabs) | | (internal Fabs) | |
| Name | VH | CH1 | Linker | VH | CH1 | Fc | VL | CL | VL | CL |
| Patri- | SEQ ID | SEQ ID | SEQ ID | SEQ ID | SEQ ID | SEQ ID | SEQ ID | SEQ ID | SEQ ID | SEQ ID |
| trastu-Fc | NO: 5 | NO: 1 | NO: 34 | NO: 9 | NO: 2 | NO: 44 | NO: 6 | NO: 3 | NO: 10 | NO: 4 |
| Trastu- | SEQ ID | SEQ ID | SEQ ID | SEQ ID | SEQ ID | SEQ ID | SEQ ID | SEQ ID | SEQ ID | SEQ ID |
| Patri-Fc | NO: 9 | NO: 2 | NO: 34 | NO: 5 | NO: 1 | NO: 44 | NO: 10 | NO: 4 | NO: 6 | NO: 3 |
| Patri- | SEQ ID | SEQ ID | SEQ ID | SEQ ID | SEQ ID | SEQ ID | SEQ ID | SEQ ID | SEQ ID | SEQ ID |
| Cetu-Fc | NO: 5 | NO: 1 | NO: 34 | NO: 7 | NO: 2 | NO: 44 | NO: 6 | NO: 3 | NO: 8 | NO: 4 |
| Cetu- | SEQ ID | SEQ ID | SEQ ID | SEQ ID | SEQ ID | SEQ ID | SEQ ID | SEQ ID | SEQ ID | SEQ ID |
| Patri-Fc | NO: 7 | NO: 2 | NO: 34 | NO: 5 | NO: 1 | NO: 44 | NO: 8 | NO: 4 | NO: 6 | NO: 3 |
| Patri- | SEQ ID | SEQ ID | SEQ ID | SEQ ID | SEQ ID | SEQ ID | SEQ ID | SEQ ID | SEQ ID | SEQ ID |
| Matu-Fc | NO: 5 | NO: 1 | NO: 34 | NO: 11 | NO: 2 | NO: 44 | NO: 6 | NO: 3 | NO: 12 | NO: 4 |
| Matu- | SEQ ID | SEQ ID | SEQ ID | SEQ ID | SEQ ID | SEQ ID | SEQ ID | SEQ ID | SEQ ID | SEQ ID |
| Patri-Fc | NO: 11 | NO: 2 | NO: 34 | NO: 5 | NO: 1 | NO: 44 | NO: 12 | NO: 4 | NO: 6 | NO: 3 |

### Gene Synthesis

The amino acid sequences of patritumab (anti-HER3), atezolizumab (anti-PDL1), cetuximab (anti-EGFR) and matuzumab (anti-EGFR) were used to design the DNA sequences, after codon optimization for mammalian expression. These antibodies are referred to as the “parental” antibodies.

The DNA construct of the heavy chain was designed as such:
signal peptide (SEQ ID NO: 79),
followed by the sequence consisting of the variable region of the external Fab followed by the constant CH1 domain of the external Fab,
followed by the linker,
followed by the variable region of the internal Fab followed by the constant CH1 domain of the internal Fab.

Flanking sequences for restriction enzyme digestion were introduced at both ends of the heavy chain DNA construct.

The DNA construct for the light chain of the external Fab was designed as such:
signal peptide (SEQ ID NO: 80),
followed by the variable region of the external Fab followed by a constant Kappa region of the external Fab.

The DNA construct for the light chain of the internal Fab was designed as such:
signal peptide (SEQ ID NO: 80),
followed by the variable region of the internal Fab followed by a constant Kappa region of the internal Fab.

All DNA constructs were synthesized by GeneWis or Eurofins. PCR reactions, using PfuTurbo Hot Start, were carried out to amplify the inserts, which were then digested with NotI and ApaI, and NotI and HindIII for heavy and light chains, respectively. The double digested heavy chain fragments were ligated with NotI and ApaI digested Icosagen's proprietary pQMCF expression vector into which the human IgG1 hinge followed by the CH2-CH3 domains were already inserted. The double-digested light chain fragments were ligated with NotI and HindIII digested Icosagen's proprietary pQMCF expression vector. Plasmid DNAs were verified by double strand DNA sequencing.

### Expression and Purification

The bispecific antibodies were produced employing transient gene expression by co-transfecting 3 genes coded on separate vectors in a 2:1:1=HC:LC1:LC2 molecular ratio (1 continuous heavy chain (HC) and 2 light chains (LC)). For a 50 mL scale expression, a total of 50 μg of plasmid DNAs in Icosagen's proprietary pQMCF vector (25 μg heavy chain+12.5 μg of each light chain, LC1 and LC2) were mixed in 1.5 mL Eppendorf tube, 1 mL of CHO TF (Xell AG) growth medium containing Icosagen's proprietary transfection Reagent 007, incubated at RT for 20 min. The mixture was loaded onto 49 mL of CHOEBNALT85 1E9 cells at 1-2×10⁶ cells/mL in 125 mL shaking flask in CHO TF (Xell AG) growth medium. Cells were shaken for 2 days at 37° C.; on day 3 the temperature was shifted to 32° C. and the cultures were fed; and on day 4 the temperature was lowered to 30° C.; the duration of complete production was 10 days. The supernatant was harvested by centrifuging cells at 3,000 rpm for 15 min. The harvested supernatants from the bispecific antibodies were purified by Protein A resin (MabSelect SuRe 5 mL column). The antibody was eluted from protein A using 0.1 M glycine pH 3.5, and the eluate was neutralized by 1 M TRIS. The bispecific antibodies further purified employing Gel Filtration Chromatography employing Superdex 200 HiLoad 26/60 pg preparative columns, whereas the Fab-Fab antibodies where purified employing Superdex 200 Increase 10/300 GL; all antibodies were buffer-exchanged into PBS pH 7.4. All samples were sterile filtered employing 0.2 μm ULTRA Capsule GF. The apparent MW was determined using Ladder Precision Plus Protein Unstained Standards (Biorad). The bispecific antibody typically exhibited good expression titer in transient CHO expression.

### SDS Polyacrylamide Gel Electrophoresis

In order to evaluate the quality of purified antibodies, we performed SDS-PAGE. In the presence of sodium dodecyl sulfate (SDS) in the running buffer, the rate at which an antibody migrates in the gel depends primarily on its size, enabling molecular weight determination. This assay was performed under non-reducing conditions and under reducing conditions; the latter permits disruption of the disulfide bonds, and hence visualization of individual polypeptide chains (the light chains and the heavy chain).

The SDS-PAGE of all bispecific antibodies exhibited the expected profiles, under both non-reducing and reducing conditions and was in agreement with the calculated theoretical molecular weights.

### Size Exclusion Chromatography Analysis

Protein aggregation is frequently observed in engineered protein molecules. We performed analytical size exclusion chromatography (SEC) to assay the high molecular weight species content of final antibody preparations after Gel Filtration Chromatography.

The SEC chromatograms demonstrated that the percentage content of higher molecular weight species is minor (less than 5%), and is similar to conventional monoclonal antibodies produced in CHO expression systems. The results confirm the absence or minor content of aggregate. The results also indicated that each antibody was correctly assembled.

### Example 2. Monospecific Binding of the Antibodies to their Cognate Antigens

The apparent affinity of BsAbs “Patri-Trastu-Fc”, “Patri-Matu-Fc” and “Patri-Cetu-Fc” for EGFR, HER2 and HER3 was assessed by direct ELISA. The monospecific binding of each BsAb, i.e. directed against one antigen at a time, is compared to the parental MAb. EC50s (antibody concentration inducing 50% median binding between baseline and maximum binding) are then defined for each BsAb and parental antibody.

### Materials and Methods

800 ng/ml of EGFR His tag, HER2 His tag or HER3 His tag antigens were diluted in 50 μl of 1×PBS in Nunc Maxisorp 96 plates. The plates were incubated overnight at 4° C., then washed 4 times with 0.1% PBS-Tween (PBS-T) and saturated with PBS-T containing 1% BSA for 1 h at 37° C. After 4 washes in 0.1% PBS-T, the antibodies (BsAbs, MAbs, and controls), were diluted in a final volume of 50 μl in 1×PBS and incubated for 1 h at 37° C. After 4 washes in 0.1% PBS-T, the detection antibody (HRP-coupled anti-human Fc) was added at 1/40000 in a 50 μl volume of 1×PBS and incubated for 1 h at 37° C. The plate was washed 4 times in 0.1% PBS-T and 50 μl of TMB was added to each well for 10-30 min at room temperature and protected from light. The reaction was stopped with 50 μl of 1M hydrochloric acid before reading the optical density at a wavelength of 450 nm.

### Results

The results are shown in Table 3 below.

TABLE 3
ELISA analysis for monospecific binding to HER3, HER2
or EGFR. Measurement of half-maximal efficient concentration
(EC50) for each parental antibody (as positive control)
or BiXAbs against HER3, HER2 or EGFR antigens

| | EC50 (nM) | | |
|---|---|---|---|
| ID | EGFR | HER2 | HER3 |
| Matuzumab | 0.09 | n.a. | n.a. |
| Cetuximab | 0.07 | n.a. | n.a. |
| Trastuzumab | n.a. | 0.09 | n.a. |
| Patritumab | n.a. | n.a. | 0.09 |
| Patri-Trastu-Fc | n.a. | 0.09 | 0.18 |
| Patri-Matu-Fc | 0.39 | n.a. | 0.16 |
| Patri-Cetu-Fc | 0.14 | n.a. | 0.19 |

The bispecific antibodies were all able to bind to the antigen and shown a binding profile very similar to the corresponding parental antibody. Sub-nanomolar apparent affinity for their cognate antigens was measured for all constructs, almost identical to their parental antibodies.

### Example 3. Dual Binding by the Bispecific Antibodies to their Cognate Antigens

Bispecific binding of the antibodies to HER3/HER2 and HER3/EGFR was assessed by an ELISA sandwich assay. Briefly, tag-free antigen was coated on the plate and incubated first with the BsAb and then the second antigen with the His-Tag; finally, an anti-HIS peroxidase antibody were used to develop the assay. This design allows to assess simultaneous binding by bispecific antibodies to both antigens.

### Materials and Methods

1000 ng/ml of EGFR, HER2 or HER3 tag free antigens were diluted in 50 μl of 1×PBS in Nunc Maxisorp 96 plates. The plates were incubated overnight at 4° C., then washed 4 times in 0.1% PBS-Tween and saturated with PBS-T containing 1% BSA for 1 h at 37° C. After 4 washes in 0.1% PBS-T, the antibodies (BsAbs or IRR), were added at various dilutions in a final volume of 50 μl of 1×PBS and incubated for 1 h at 37° C. After 4 washes in 0.1% PBS-T, 1000 ng/ml EGFR His tag, HER2 His tag or HER3 His tag antigens in 50 μl of PBS1× were incubated for 1 h at 37° C. The plates were washed 4 times with 0.1% PBS-T and HRP-coupled anti-His tag detection antibody was added at 1/2000 in 50 μl of 1×PBS. The plates were incubated for 1 h at 37° C. The plates were washed 4 times in 0.1% PBS-T and 50 μl of TMB developing solution was added for 10-30 min at room temperature, protected from light. The reaction was stopped with 50 ul of 1M hydrochloric acid before reading the optical density at a wavelength of 450 nm.

### Results

The binding curves of FIG. 2(A) show simultaneous binding profile of Patri-Trastu-Fc to immobilized HER3 developed by soluble HER2-His (left panel), and to immobilized HER2 developed by soluble HER3-His (right panel) in a dose-dependent manner. The binding curve of FIG. 2(B) shows simultaneous binding of Patri-Matu-Fc to immobilized HER3-Fc and EGFR-His (left panel), and to immobilized EGFR-Fc and HER3-His (right panel). This is also the case for the binding curves shown in FIG. 2(C) for the Patri-Cetu-Fc.

The results thereby show that the BsAbs of the invention are able to simultaneously bind to both antigens.

### Example 4. Bispecific Antibodies Inhibit Phosphorylation of AKT and ERK

The main anti-tumoral effects of antibodies targeting HER family of receptors are their ability to inhibit the signaling associated with those receptors. We thus investigated the two main signaling pathways linked to EGFR, HER2 and HER3 signaling: MAPK/ERK and PI3K/AKT. Using HTRF technique, we assessed the phosphorylation inhibition of pERK and pAKT induced by the addition of bispecific antibodies after stimulation with EGF+NRG1 ligands.

### Materials and Methods

At Day 0, 50,000 cells per wells were seeded in 100 μl of complete medium (DMEM or RPMI 10% SVF) in 96-well flat-bottom plates. At Day 1, cells were depleted of FCS by changing the 10% FCS culture medium to 2% FCS medium. At Day 2, culture medium was removed for all conditions and cells were treated with 70 nM of antibody (BsAbs or MAbs) diluted in 100 μl of 2% FCS culture medium for 20 min, to which two ligands, EGF at 16.6 nM (100 ng/ml) and NRG1 at 3.71 nM (100 ng/ml), were added for an additional 10 min at 37° C. At the end of the 30 min treatment, the medium was then removed and the cells were rinsed once with cold 1×PBS. The cells were then lysed for 1 h under agitation and pERK and pAKT levels were measured with the HTRF kit pERK Thr202/Tyr204 (CisBio®, #64ERKPEG) and pAKT Ser473 (CisBio®, #64AKSPET) according to manufacturer's recommendations. Plates were then read on the Pherastar reader (BMG LabTech) at 665 nm and 620 nm.

### Results

For the three PDAC cell lines tested (BxPC-3, AsPC-1, and CFPAC), all BsAbs (Patri-Trastu-Fc, Patri-Cetu-Fc and Patri-Matu-Fc) demonstrated strong inhibition of pAKT and PERK signaling after ligand induction (FIG. 3B). Both EGFR/HER3 bispecific antibodies blocked almost 100% of basal pAKT signaling in BxPC-3 cells and about 85-90% in ASPC1 and CFPAC cells similar to HER2/HER3 antibodies. They also efficiently inhibited pERK signaling in BxPC3 cells (85 and 99% for Patri-Matu-Fc and Patri-Cetux-Fc, respectively, compared to 50% for the Patri-Trastu-Fc). All antibodies inhibited the strongest signaling pathways in the BxPC3 cell line since this cell line was the only one expressing WT Kras, while pERK inhibition was weaker in ASPC1 and CFPAC, which expressed mutated Kras. In all three cell lines, EGFR density was much higher than that for HER2 or HER3 (FIG. 3A). Superiority of anti-EGFR-containing BiXAbs was also expected compared to anti-HER2/HER3 antibodies.

### Example 5. In Vivo Effects of Patri-Trastu-Fc, Patri-Matu-Fc and Patri-Cetu-Fc

The tumor growth and survival of bispecific antibody-treated mice xenografted with Sw1990 and PDX P2846 PDAC cells, were evaluated. Those models express mutated Kras and similar antigenic densities of EGFR, HER2 or HER3 antigens.

### Materials and Methods

At Day 0, 5-week-old Swiss nude immunodeficient mice were transplanted subcutaneously with 3 millions of Sw-1990 cells (n=9 mice/group). Treatments started when the tumors were 90 mm3 on average. Mice were treated with 17 mg/kg of bispecific antibodies administered intraperitoneally in 200 ul injections. The treatment schedule was 2 injections per week for 4 weeks. Tumor size was measured with a caliper. Mice were sacrificed when the tumor size reaches 1500 to 2000 mm3.

P2846 P7 with a size of 150 mm3 were transplanted intrascapularly under the brown body. The treatment regimen remained the same as for the Sw-1990 mouse model.

### Results

In mice xenografted with Sw1990 cells, tumor growth was inhibited by 42%, 62% and 69% with Patri-Trastu-Fc, Patri-Matu-Fc and Patri-Cetu-Fc, respectively (FIG. 4A).

Survival of Sw-1990 xenografted mice was improved by 11 days for the Patri-Trastu-Fc treated group, 15 days for the Patri-Matu-Fc treated group and 15 days for the Patri-Cetu-Fc treated group. EGFR/HER3 BsAbs induced the strongest tumor growth inhibition (875 mm3 for 3Patri-1Matu-Fc and 3Patri-1Cetu-Fc on day 46) compared to 3Patri-2Trastu-Fc EGFR/HER2 BsAb (1200 mm3 on day 46). BsAb 3Patri-1Cetu-Fc provided better overall survival. No signs of toxicity or weight loss were noted in this experiment. The negative control vehicle vs irrelevant Bispecific antibody (BsAb IRR) showed similar tumor growth and survival.

In mice xenografted with PDX P2846, tumor growth was inhibited by 87% with Patri-Cetu-Fc, by 77% with Patri-Trastu-Fc and by 63% with Patri-Matu-Fc (45 days post-graft) (FIG. 4B).

On day 80, the survival of xenografted mice with P2846 P7 PDX was improved by 70% for Patri-Trastu-Fc and Patri-Cetu-Fc treated groups and by 20% for the Patri-Matu-Fc treated group compared with the BsAb IRR group. BsAbs Patri-Trastu-Fc and Patri-Cetu-Fc induced the strongest tumor growth inhibition compared to Patri-Matu-Fc. Survival was improved by 34 days in Patri-Trastu-Fc group and 41 days in Patri-Cetu-Fc group compared to the control BsAb IRR group. No signs of toxicity or weight loss were noted in this trial.

### Example 6. Intratumoral Penetration of BsAb in Sw-1990 Xenografts

A common question concerning large molecular weight of BsAbs (250 kDa) relative to conventional antibodies (150 kDa) concerns their ability to penetrate into tumors, particularly into pancreatic tumors known to have abundant and dense stroma. Histological sections were made from the extracted tumors; Sw1990 tumor penetration by the bispecific antibodies was analyzed by immunohistochemistry. Bispecific antibodies were labelled using peroxidase-conjugated anti-human Fc. BsAb IRR was a control bispecific antibody targeting CD19 and CD3. NaCl was used as negative control (FIG. 5).

Specific labeling of BsAbs was observed in tumors treated with Patri-Trastu-Fc, Patri-Matu-Fc and Patri-Cetu-Fc. The image at 40× showed that the labeling is homogeneous between the center of the tumor and the periphery, despite the dense stroma, suggesting that the BsAbs penetrate the tumor. In addition, a pronounced labeling is observed at the membrane of tumor epithelial cells in the center of the tumor, demonstrating binding and accumulation of the BsAbs at the membrane of tumor cells in the interior of the tumor mass. No non-specific labeling was observed for tumors treated with NaCl. BsAb IRR is weakly detected at the tumor periphery or stroma, and is not detected at the tumor epithelial cells, indicating that BsAb IRR does not bind to tumor cells.

### Example 7. Patri-Trastu-Fc, Patri-Matu-Fc and Patri-Cetu-Fc Decrease Angiogenesis

Angiogenesis or neovascularization has been studied using anti-CD31 staining by immunohistochemistry (IHC). CD31 is expressed on endothelial cells and thus allows the labeling of blood vessels. Angiogenesis is a major parameter in tumor transformation, especially in pancreatic cancer. The proportion of blood vessels was evaluated after a treatment with the 3 BsAbs compared to controls (NaCl and BsAb IRR). For each treatment, 3 tumor sections corresponding to three different mice were labeled and the images were analyzed by ImageJ software. The results of the quantification are represented in histograms highlighting the variation in vessel size according to the treatments (FIG. 6A).

The IHC results demonstrate a decrease in blood vessel size from 75 μm for the control groups (NaCl, BsAb IRR) to 55 μm for the groups treated with the BsAbs of the invention. This suggests inhibition of xenograft neovascularization by BsAbs.

### Example 8. Ex Vivo Analysis of NK Infiltration in the Tumor Treated by Patri-Trastu-Fc, Patri-Matu-Fc and Patri-Cetu-Fc

### Materials and Methods

NK cell immunophenotyping was conducted by flow cytometry on resected Sw1990 xenografts from BsAb-treated mice. Dissociated cells were labeled with Live/dead, mouse CD45, CD3, CD19, CD49, NKp46, IFNγ and CD1070-specific antibodies. NK cells were isolated for immunophenotyping using a panel containing phycoerythrin-conjugated anti-CD3, and APC-Cy7-conjugated anti-CD19 antibodies for negative selection, and AF700-conjugated anti-CD45, APC-conjugated anti-CD49b, fluorescein-conjugated anti-NKp46 for positive selection. Activation of CD45+CD49b+ NKp46+NK cells was analyzed by flow cytometry after BV786-conjugated anti-CD107a, and intracellular BV421-conjugated anti-IFNg labelling.

### Results

The NK infiltration increased by 2-fold in tumors treated with the BsAbs of the invention compared to the NaCl and BsAb IRR groups, as shown in the CD49+NKp46+ analysis (FIG. 5B). The increase of IFNγ+ and CD107a+ on NK cells suggested that these intra-tumoral NK cells are also activated compared to untreated tumors and thus could mediate the ADCC.

### Example 9. Ex Vivo Efficacy of Patri-Trastu-Fc, Patri-Matu-Fc and Patri-Cetu-Fc on Egfr, HER2 and HER3 Receptor Degradation

### Materials and Methods

EGFR staining from Patri-Cetu-Fc-vs BsAb IRR- and NaCl-treated Sw1990 xenograft section was carried out. HER receptor expression was determined by Western blot analysis for each Sw1990 xenograft extract treated with NaCl, Patri-Trastu-Fc, Patri-Matu-Fc and Patri-Cetu-Fc respectively. Tubulin was used as loading control. Western blots were visualized using primary rabbit monoclonal antibodies against EGFR, HER2, and HER3, before adding the secondary IRDye800-conjugated goat anti-rabbit IgG (1/20000 dilution). The fluorescence was quantified using the LI-COR Odyssey imaging system. HER receptor expression was then quantified and normalized to tubulin, in each group of resected xenografts. Each experiment was performed on three Sw1990-xenografted mice. BsAb IRR is a negative control which binds simultaneously to human CD19 and CD3.

### Results

The images obtained at ×20 and ×40 magnifications (FIG. 5C, top panel) showed a clear decrease in EGFR signal in the tumor treated with Patri-Cetu-Fc compared to the tumor treated with NaCl or BsAb IRR.

EGFR, HER2, and HER3 total protein levels were assessed by western blot on protein extracts from Sw-1990 xenografts and revealed by immunofluorescence. Total protein levels were quantified and normalized to tubulin. The results of FIG. 5C (middle and upper panel) show that the 3 BsAbs of the invention induce EGFR, HER2 or HER3 degradation compared to the NaCl and BsAb IRR groups.

### Example 10. The Bispecific Antibody Patri-Cetu-Fc More Potently Decreased Tumor Growth Compared to Monospecific Antibodies or their Combination in Sw1990 Xenograft Model

### Materials and Methods

Mice xenografted with Sw1990 cells were treated with (1) BisAb IRR anti-CD19-CD3 (2) bispecific antibody Patri-Cetu-Fc (17 mg/Kg) (3) Cetuximab parental antibody (10 mg/kg) (4) Patritumab parental antibody (10 mg/kg) or the combination (Cetuximab+Patritumab, 5+5 mg/kg) (9 mice per group). Doses of the bispecific antibody and parental antibodies possessed identical molarity based on their Fc-content.

At Day 0, Swiss nude immunodeficient mice were transplanted subcutaneously with 3 millions of Sw-1990 cells. Treatments started when the tumors have an average size of 140 mm3. The treatment schedule was 2 injections per week for 4 weeks. Tumor growth and survival of the treated mice were evaluated (FIG. 7).

### Results

BsAb Patri-Cetu-Fc induced a strong inhibition of tumor growth in vivo (77% of tumor growth inhibition on day 38 post-graft compared to IRR-treated mice). Monospecific antibodies, Cetuximab and Patritumab induced a tumor growth inhibition of 50% and 43% respectively, on day 38 post-graft. Finally, the combination of the two monoclonal antibodies showed 65% of tumor growth inhibition when compared to IRR-treated mice. The bispecific antibody Patri-Cetu-Fc is thus much more potent than monospecific parental antibodies, in terms of tumor growth inhibition.

Patri-Cetu-Fc also induced a strong survival benefit in vivo. The bispecific antibody Patri-Cetu-Fc was shown to be more potent than monospecific parental antibodies, in terms of survival benefit.

### Example 11

Two additional bispecific antibodies comprising a humanized Cetuximab Fab, were designed:
The “BMX003-010” antibody, which comprises Patritumab Fab domain (as external Fab) and a humanized version of Cetuximab Fab domain (as internal Fab).
The “BMX003-011” antibody, which is identical to BMX003-010, except that it further comprises 2 mutations in the Fc domain (S239D and 1332E).

BMX003-010 and BMX003-011 both comprise a humanized Cetuximab Fab comprising:
VH domain of SEQ ID NO: 81 and CH1 domain of SEQ ID NO: 2 (corresponding to VH-CH1 sequence of SEQ ID NO: 83),
VL domain of SEQ ID NO: 71 and CL domain of SEQ ID NO: 4 (corresponding to VL-CL sequence of SEQ ID NO: 84).

BMX003-011 differs from BMX003-010 in that it comprises 2 mutations (S239D and 1332E) in the Fc domain (Mutated Fc domain of SEQ ID NO: 82).

TABLE 4
details on the sequences of bispecific construct “BMX003-
010” and “BMX003-011”.

| | | Light | Light |
|---|---|---|---|
| | | chains | chains |
| | Heavy | (external | (internal |
| Name | chains | Fabs) | Fabs) |
| BMX003-010 | SEQ ID NO: 85 | SEQ ID NO: 14 | SEQ ID NO: 84 |
| BMX003-011 | SEQ ID NO: 86 | SEQ ID NO: 14 | SEQ ID NO: 84 |

The activity of BMX003-010 and BMX003-011 that comprise a humanized Cetuximab Fab fragment, was compared to the activity of “BMX003-001” that comprises parental Cetuximab Fab (“BMX003-001” corresponds to the “Patri-cetu-Fc” construct described above).

### Sds Polyacrylamide Gel Electrophoresis and Size Exclusion Chromatography Analysis

In order to evaluate the quality of purified antibodies, a SDS-PAGE and size exclusion chromatography (SEC) were performed as described above. The SDS-PAGE of BMX003-010 and BMX003-011 bispecific antibodies exhibited the expected profiles. The SEC chromatograms confirmed the absence or minor content of aggregate and indicated that each antibody was correctly assembled.

### Affinity for HER3 and EGFR Assessed by ELISA

The apparent affinity of BsAbs BMX003-010, BMX003-011 and BMX003-001 (Patri-Cetu-Fc) antibodies for HER3 and EGFR was assessed by direct ELISA. The monospecific binding of each BsAb, i.e. directed against one antigen at a time, is compared to the parental MAb. EC50s (antibody concentration inducing 50% median binding between baseline and maximum binding) are then defined for each BsAb and parental antibody. ELISA assay was carried out as described above.

FIGS. 8 and 9 show that the bispecific antibodies were all able to bind to their antigens (HER3 and EGFR, respectively), with a binding profile very similar to the corresponding parental antibody.

### Flow Cytometry Binding Profile

The Ability of BMX003-010, BMX003-011, and BMX003-001 (Patri-Cetu-Fc), to bind to the HER3 and EGFR proteins expressed on the cell surface of SW-1990 cells, was measured by flow cytometry (n=3).

The results of FIG. 10 show that BMX-003-001, and humanized BMX-003-010 and BMX-003-011 have very similar EC50 and ECmax.

### Phosphorylation Assay

Based on the Advanced Phospho-ERK and Advanced Phospho-AKT kits from Perkin Elmer. HCT-116 colon cancer cells were plated in cDMEM media at a density of 50,000 cells/well in a 96 well. Flat bottomed micro-titre plate and grown over night at 37° C. (5% CO2). Tissue culture media was removed and replaced with serum-free media (DMEM+GlutaMAX) and the cells were incubated for a further 24 hrs at 37° C. (5% CO2). Tissue culture media was removed and replaced with test articles (BiXAbs, MAbs or controls) resuspended in DMEM and incubated for 20 mins at 37° C. (5% CO2). Growth factors (16.6 nM EGF and 3.71 nM NRG1) were added to the wells and incubated for a further 10 mins. Medium was removed and cells were then washed once with cold PBS. Cells were then lysed by the addition of Lysis buffer (provided in kit from Perkin Elmer). Quantitation of the phosphor-signal was achieved by following the FRET assays kit instructions provided by Perkin Elmer. In essence, 4 microL of premixed europium labelled antibodies were incubated with the cell lysates for 4 hrs at room temperature. Supernatants were then transferred to a fluorescence plate reader and HTRF was measured at 665 nm and 620 nm.

The results show that all bispecific antibodies demonstrated strong inhibition of pAKT (FIG. 11 A) and pERK (FIG. 11 B) signaling after ligand induction.

### Degranulation Assay

PBMCs from two independent donors were prepared following standard procedures and rested overnight in complete RPMI media The HCT116 colon cancer cell line was grown in RPMI tissue culture media supplemented with 10% FBS. 20,000 HCT116 cells were plated in U-bottomed 96 well micro-titre plates together with 100,000 PBMCs (giving an Effector to Target (E:T) ratio of 1:2 based on the NK population being approximately 10% of the PBMCs). BiXAb test articles and control were then added to the cell mixture together with the anti-CD107a-AF488 mAb and incubated for 1 hr at 37° C. (5% CO2). GolgiStop was then added to the wells and the plates were incubated for a further 4 hrs. Anti CD45, anti-CD3 and CD56 mAbs were then added to the wells to permit the identification of NK cells by typical FACs staining. NK degranulation was assessed by FACS analysis of CD107a by gating on NK cells.

The results of FIG. 12 show that Cetuximab, BMX003-001 and BMX003-010 are comparable. A much higher degranulation was observed in presence of BMX003-011 (Fc-modified), suggesting that the BMX003-011 mutated Fc domain has an increased binding to Fc gamma receptors when compared to wild-type Fc domain, leading to a bispecific antibody having an improved effector function.

### In Vivo Efficacy

In vivo efficacy of BMX003-001 (Patri-Cetu-Fc), BMX003-010 (humanized Patri-Cetu-Fc), and BMX003-011 (humanized Patri-Cetu-Fc modified) was assessed in a SW1990 pancreatic cancer xenograph model. BMX003-01, BMX003-010 and BMX003-011 were used at 8.5 mg/kg and BMX control at 17 mg/kg. The method is as described above.

On Day 35 (after the last injection on Day 32), all the groups treated with HER3×EGFR BiXAbs presented significantly lower tumor volumes than the control group PBS. No major differences between BMX003-001, BMX-003-010 and BMX-003-011 BiXAbs was observed.

### CONCLUSION

The results show that the bispecific antibodies of the invention were efficiently produced, and properly assembled. The results also demonstrate the therapeutic properties of the bispecific antibodies:
simultaneously bind to their targets with good affinity when compared to parental antibodies;
inhibit the activation of HER3/HER2/EGFR receptors, by inhibiting the phosphorylation signaling pathway;
simultaneously degrade the two targeted receptors;
penetrate into the tumor;
inhibit the neovascularization;
modulate the infiltration of immune cells in the tumor; and
significantly inhibit tumor growth and improve survival in vivo.

1.-14. (canceled)

15. A bispecific antigen-binding fragment which is capable of simultaneous binding to HER-3 and to another antigen selected from HER-2 and EGFR antigens, which comprises:
(i) a Fab fragment comprising a VH-CH1 heavy chain associated with a VL-CL light chain of antibody 1 (Ab1), and
(ii) a Fab fragment comprising a VH-CH1 heavy chain associated with a VL-CL light chain of antibody 2 (Ab2),
wherein the N-terminal end of the VH domain of the Fab fragment of Ab1 is linked to the C-terminal end of the CH1 domain of the Fab fragment of Ab2 through a polypeptide linker,
wherein one of Ab1 or Ab2 is patritumab or a functional derivative thereof;
and the other of Ab1 or Ab2 is selected from the group consisting of trastuzumab or a functional derivative thereof, matuzumab or a functional derivative thereof, and cetuximab or a functional derivative thereof.

16. The bispecific antigen-binding fragment of claim 15, wherein Ab2 is patritumab or a functional derivative thereof and wherein Ab1 is selected from the group consisting of trastuzumab or a functional derivative thereof, matuzumab or a functional derivative thereof, and cetuximab or a functional derivative thereof.

17. The bispecific antigen-binding fragment of claim 15, wherein the functional derivative of cetuximab is a humanized form comprising VH and VL chain amino acid sequences at least 80% identical to VH and VL chain amino acid sequences of cetuximab, respectively.

18. The bispecific antigen-binding fragment of claim 15, wherein the CH1 and CL domains of Ab1 have a sequence different from the CH1 and CL domains of Ab2.

19. The bispecific antigen-binding fragment of claim 15, wherein the Fab CH1 domain of one of Ab1 or Ab2 is a mutated domain that derives from the CH1 domain of an immunoglobulin by substitution of the threonine residue at position 192 of said CH1 domain with a glutamic acid and the cognate CL domain is a mutated domain that derives from the CL domain of an immunoglobulin by substitution of the asparagine residue at position 137 of said CL domain with a lysine residue and substitution of the serine residue at position 114 of said CL domain with an alanine residue, and/or wherein the Fab CH1 domain of one or the other of Ab1 or Ab2 is a mutated domain that derives from the CH1 domain of an immunoglobulin by substitution of the leucine residue at position 143 of said CH1 domain with a glutamine and substitution of the serine residue at position 188 of said CH1 domain with a valine residue, and the cognate CL domain is a mutated domain that derives from the CL domain of an immunoglobulin by substitution of the valine residue at position 133 of said CL domain with a threonine residue and substitution of the serine residue at position 176 of said CL domain with a valine residue.

20. The bispecific antigen-binding fragment of claim 15, wherein the polypeptide linker sequence comprises: EPKX1CDKX2HX3X4PPX5PAPELLGGPX6X7PPX8PX9PX10GG (SEQ ID NO:33), wherein X1, X2, X3, X4, X5, X6, X7, X8, X9, X10, identical or different, are any amino acid.

21. A bispecific molecule that comprises two identical antigen-binding arms, each consisting of an antigen-binding fragment as defined in claim 15.

22. The bispecific molecule of claim 21, which is a full-length antibody comprising two heavy chains and four light chains,
wherein each heavy chain comprises
a) a Fc region of an immunoglobulin comprising Hinge-CH2-CH3 domains;
b) which Fc region is linked to the Fab VH-CH1 heavy chain of Ab1 by said hinge domain, wherein the hinge domain links the N-terminal end of said CH2 domain with the C-terminal end of CH1 domain of Ab1;
c) which in turn is linked to Fab VH-CH1 heavy chain of Ab2, by the polypeptide linker sequence, wherein the polypeptide linker sequence links the N-terminal end of said VH domain of Ab1 with the C-terminal end of said CH1 domain of Ab2,
and wherein the four light chains comprise two Fab VL-CL light chains of Ab1 and two Fab VL-CL light chains of Ab2 associated with their cognate heavy chain domains.

23. The bispecific molecule of claim 21, which comprises a) two heavy chains, each comprising SEQ ID NO: 27 and b) four light chains, two comprising SEQ ID NO: 14, the two others comprising SEQ ID NO: 18.

24. The bispecific molecule of claim 21, which comprises a) two heavy chains, each comprising SEQ ID NO: 29 and b) four light chains, two comprising SEQ ID NO: 14, the two others comprising SEQ ID NO: 16.

25. The bispecific molecule of claim 21, which comprises a) two heavy chains, each comprising SEQ ID NO: 31 and b) four light chains, two comprising SEQ ID NO: 14, the two others comprising SEQ ID NO: 20.

26. A method for producing a bispecific molecule, said method comprising the following steps:
a) culturing in suitable medium and culture conditions a host cell expressing the bispecific molecule as defined in claim 22, and
b) recovering said produced bispecific molecule from the culture medium or from said cultured cells.

27. A method of treating a subject comprising administering a bispecific antigen-binding fragment as defined in claim 15 to said subject.

28. A method of treating a patient suffering from cancer comprising administering a bispecific antigen-binding fragment as defined in claim 15 to said patient.

29. The method of claim 28, wherein the cancer is a solid tumor.

30. The method of claim 28, wherein the cancer is a pancreatic cancer, head and neck cancer, colorectal cancer, breast cancer, or lung cancer.
